(19) United States
(12) Patent Application Publication
(10) Pub. No.: US 2023/0210746 A1
(43) Pub. Date: Jul. 6, 2023

(54) ANTI-AGING COSMETIC COMPOSITIONS COMPRISING NMN

(71) Applicant: NUVAMID SA, Lausanne (CH)

(72) Inventors: Guillaume BERMOND, MARSEILLE (FR); Laurent GARCON, SAUSSET LES PINS (FR)

(73) Assignee: NUVAMID SA, Lausanne (CH)

(21) Appl. No.: 17/926,246

(22) PCT Filed: May 19, 2021
(86) PCT No.: PCT/EP2021/063312

§ 371 Date: Nov. 18, 2022

(30) Foreign Application Priority Data
May 20, 2020 (FR) FR2005287
Mar. 31, 2021 (FR) FR2103340
Mar. 31, 2021 (FR) FR2103342

(51) Int. Cl. A61K 8/60; A61Q 19/08; A61K 8/9789; A61K 8/73

(52) U.S. Cl. CPC A61K 8/606; A61Q 19/08; A61K 8/9789; A61K 8/735

(57) ABSTRACT

The invention pertains to a cosmetic composition comprising NMN, a precursor thereof, a derivative thereof, against skin ageing.

### FIELD OF THE INVENTION

The present invention pertains to cosmetic compositions comprising nicotinamide mononucleotide (NMN), a derivative thereof, a precursor thereof or a salt thereof, for cosmetic purposes to prevent and/or treat at least one sign of ageing.

### TECHNICAL BACKGROUND

The skin is an organ covering the entirety of the human body. It has the role of forming a barrier between the body and the outside environment, and hence in particular of protecting the body against external harm, of maintaining body temperature. The skin is composed of several layers the outermost being the epidermis.

From the innermost layer to the outermost layer, the epidermis comprises the basal cell layer (stratum germinativum), then the spiny layer (stratum spinosum), then the granular layer (stratum granulosum) composed of 3 to 5 layers of flattened cells, followed by the translucent or clear layer (stratum lucidum) which is a very thin area, and finally the cornified layer (stratum corneum) composed of flat keratinized anucleate cells. The stratum corneum is composed of lines of flattened cells chiefly composed of keratin. These are squamous cells. These cells are shed in layers every day.

Ageing of the skin deteriorates several skin parameters such as the firmness, elasticity, and density thereof. Wrinkles can form on the skin surface. The eye contour can also show dark circles and swelling of the lower eyelid. All these elements can be of unsightly appearance.

Ageing of the skin can have a different effect on subjects depending on skin type. There are different skin types: normal skin, dry skin, oily skin and mixed skin types having areas of normal skin and areas of oily skin, or a combination of dry and oily skin.

Healthy skin is naturally protected on the surface by a hydrolipid film chiefly formed of water and lipids (sebum). Being impermeable, it protects the skin against external harm and prevents water loss. When the upper layer of the epidermis does not have water/lipid balance, it can no longer efficiently fulfil its barrier role and becomes uncomfortable to varying extents. The subject experiences feelings of tightness, burning or itching when the skin becomes dry. Dry skin has various causes. Unlike dehydrated skin, dry or very dry skin is a chronic condition generally due to dysfunction of genetic origin. In addition to genetic origin, there also exist conditions of skin dryness triggered by external factors such as weather or environmental factors (pollution, air conditioning, room heating). Skin dryness can also be caused by skin pathologies, diseases affecting the whole body such as thyroid dysfunction, diabetes, dietary deficiency and/or medical treatment such as anti-acne treatments.

Dry skin is characterized by the following symptoms: dull appearance, particular epidermal clinical signs of desquamation, cracking, at times inflammation and loss of elasticity. Irregularities of the skin surface are greater the dryer the skin becomes. Dry skin provides a favourable environment for the onset of eczema, in particular in the form of eczematid achromians. There are different types of dry skin: (i) dry skin characterized by transient discomfort, slight tightness and/or slight flaking; (ii) very dry skin having marked tightness and discomfort, intense flaking, cracking and chapping; and (iii) pre-atopic xerosis characterized by rough, coarse skin and slight irritation.

Oily skin is characterized by hypersecretion of sebum by the seborrheic glands of the epidermis. Oily skin can particularly exhibit imperfections such as spots and/or blackheads, dull complexion, shiny appearance of the skin. The causes of oily skin are genetic first of all. However, sebum hypersecretion can also be caused by hormonal influence, the subject's gender, environmental pollution, high outdoor temperatures, diet too high in fat, stress and other factors.

Normal skin is characterized by normal production of sebum and does not show the characteristics of dry skin.

Mixed skin is characterized by oily skin restricted to some areas of the face, and in particular the forehead, nose and/or chin.

The cosmetic industry proposes numerous cosmetic compositions to solve this problem. However, individuals can develop allergies to conventional compositions, in particular compositions comprising retinol a cosmetic ingredient used to treat skin ageing.

There is therefore a need to develop novel cosmetic compositions to prevent or treat signs of ageing, reducing the risk of reaction.

### SUMMARY OF THE INVENTION

These objectives are achieved by means of cosmetic compositions comprising nicotinamide mononucleotide (NMN), a derivative thereof, a precursor thereof or a salt thereof, for cosmetic use thereof to prevent and/or treat at least one sign of ageing.

Advantageously, the at least one sign of ageing can be selected from among wrinkles, an increase in skin roughness, reduced thickness of the epidermis or dermis, reduced firmness of the epidermis and dermis, reduced elasticity of the epidermis and dermis, diminished skin radiance, swelling of the lower eyelid, erythema of the skin under the eyes, dark circles, and combinations thereof.

Advantageously, the composition of the invention can prevent or treat at least one sign of ageing of dry skin.

Advantageously, the composition of the invention can prevent or treat at least one sign of ageing of normal skin.

In one embodiment, the derivative of NMN can be selected from among alpha nicotinamide mononucleotide (α-NMN), dihydronicotinamide mononucleotide (denoted NMN-H), the compound of formula (I):

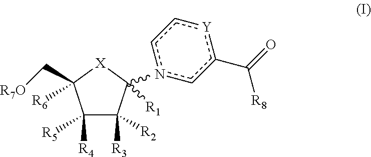

or one of the pharmaceutically acceptable stereoisomers, salts, hydrates, solvates or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ et R₅ are each independently selected from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHRAANH₂; wherein RAA is a side chain selected from among a proteinogenic amino acid;
R₆ is selected from among H, azido, cyano, C₁-C₈ alkyl, C₁-C₈ thio-alkyl, C₁-C₈ heteroalkyl and OR; wherein R is selected from H and (C₁-C₈) alkyl;
R₇ is selected from among H, P(O)R₉R₁₀, P(S)R₉R₁₀ and

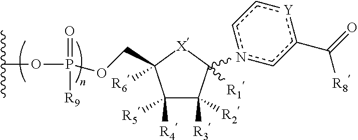

wherein n is an integer equal to 1 or 3; in which
R₉ and R₁₀ are each independently selected from among OH, OR₁₁, NHR₁₃, NR₁₃R₁₄, a (C₁-C₈) alkyl, a (C₂-C₈) alkenyl, a (C₂-C₈) alkynyl, a (C₃-C₁₀) cycloalkyl, a (C₅-C₁₂) aryl, (C₁-C₈) alkyl aryl, (C₁-C₈) aryl alkyl, (C₁-C₈) heteroalkyl, (C₁-C₈) heterocycloalkyl, a heteroaryl, and NHCHR_{A}R_{A}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)nC(O)(C₁-C₁₅)alkyl, —(CH₂)nOC(O)(C₁-C₁₅)alkyl, —(CH₂)nOC(O)O(C₁-C₁₅)alkyl, —(CH₂)nSC(O)(C₁-C₁₅)alkyl, —(CH₂)nC(O)O(C₁-C₁₅)alkyl, and —(CH₂)nC(O)O(C₁-C₁₅)alkyl aryl; wherein n is an integer selected from 1 to 8; and P(O)(OH)OP(O)(OH)₂, halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂ SO² wherein each of R₁₁ₐ is independently selected from among H and (C₁-C₆) alkyl and R_{11b} is independently selected from among OH, C₁-C₆ alkoxy, NH₂, NH(C₁-C₆ alkyl) or N(C₁-C₆ alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH2)3NHC(═NH)NH2, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among a hydroxyl, (C₁-C₁₀) alkyl, (C₁-C₆) alkoxy, a halogen, a nitro, and a cyano; or
R₉ and R₁₀, together with the phosphorus atoms to which they are attached, form a 6-membered ring in which —R₉-R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
   R₉ and R₁₀, together with the phosphorus atoms to which they are attached, form a 6-membered ring in which —R₉-R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃ and halogen; wherein R₁₃ and R₁₄ are each independently selected from among H, (C₁-C₈) alkyl and (C₁-C₈) alkyl aryl; and
R₁₃ and R₁₄ are each independently selected from among H, C₁-C₈ alkyl and C₁-C₈ alkyl aryl;
R₁₅ and R₁₆ are independently selected from among H, C₁-C₈alkyl and C₁-C₈alkyl aryl; and —CR_{B}R_{C}—C(O)—OR_{D} in which R_{B} and R_{C} are independently selected from among H, C₁-C₆ alkyl, C₁-C₆ alkoxy, benzyl, indolyl or imidazolyl, wherein the groups C₁-C₆ alkyl and C₁-C₆ alkoxy can optionally and each independently be substituted by one or more from among a halogen, an amino, amido, guanidyl, hydroxyl, thiol or carboxyl group, and the benzyl group is optionally substituted by one or more from among a halogen or hydroxyl group, or R_{B} and R_{C} together with the carbon atom to which they are attached form a C₃-C₆ cycloalkyl group optionally substituted by one or more from among a halogen, an amino, amido, guanidyl, hydroxyl, thiol or carboxyl group, and R_{D} is H, C₁-C₆ alkyl, C₂-C₆ alkenyl, C₂-C₆ alkynyl or C₃-C₆ cycloalkyl;
Y is selected from among CH, CH₂, C(CH₃)₂ and CCH₃;
[symbol] represents a single or double bond depending on Y; and
[symbol] represents the alpha or beta anomer depending on the position of R₁
   or one of the stereoisomers, one of the salts, one of the hydrates, one of the solvates or one of the crystals thereof.
   or
   the compound of formula (Ia):

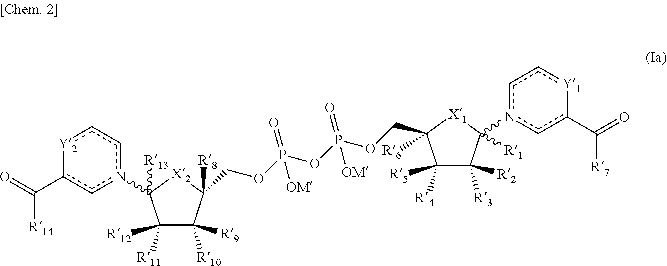

or one of the stereoisomers, one of the salts, one of the hydrates, one of the solvates, or one of the crystals thereof, in which:
X′₁ and X′₂ are independently selected from among O, CH₂, S, Se, CHF, CF₂, and C═CH₂;
R′₁ and R′₁₃ are independently selected from among H, azido, cyano, a C₁-C₈ alkyl, a C₁-C₈ thio-alkyl, C₁-C₈ heteroalkyl, and OR, wherein R is selected from among H and C₁-C₈ alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH₂ group; wherein R_{AA} is a side chain selected from a proteogenic amino acid;
R′₆ and R′₈ are independently selected from among H, an azido, a cyano, a C₁-C₈ alkyl and OR, wherein R is selected from among H and a C₁-C₈ alkyl;
R′₇ and R′₁₄ are independently selected from among H, OR, NHR, NRR′, NH—NHR, SH, CN, N3 and a halogen, wherein R and R′ are independently selected from among H and a (C₁-C₈) alkyl aryl;
Y′₁ and Y′₂ are independently selected from among CH, CH₂, C(CH₃)₂ or CCH³;
M′ is selected from among H or a suitable counter ion; [symbol]
represents a single or double bond depending on Y′1 et Y′2; and [symbol] represents an alpha or beta anomer depending on the position of R′1 and R′13;
and combinations thereof.

In a first preferred embodiment, the derivative is the compound having formula (I).

In one variant of the first embodiment, X represents an oxygen.

In one variant of the first embodiment, R₁ and R₆ are each independently a hydrogen.

In one variant of the first embodiment, R₂, R₃, R₄ and R₅ are each independently a hydrogen or OH.

In one variant of the first embodiment, Y is CH.

In one variant of the first embodiment, Y is CH₂.

In one variant of the first embodiment, R₇ is a hydrogen.

In one variant of the first embodiment, R₇ is P(O)(OH)₂.

In one variant of the first embodiment, X represents an oxygen; and/or R₁ and R₆ each independently represent a hydrogen; and/or R₂, R₃, R₄ and R₅ each independently represent hydrogen or R₂, R₃, R₄ and R₅ independently represent OH; and/or Y represents a CH our CH₂; and/or R₇ represents P(O)R₉R₁₀, wherein R₉ and R₁₀ are independently selected from among OH, OR₁₁, NHR₁₃, NR₁₃R₁₄, C₁-C₈alkyl, C₂-C₈ alkenyl, C₂-C₈ alkynyl, C₃-C₁₀ cycloalkyl, C₅-C₁₂ aryl, C₁-C₈ aryl alkyl, C₁-C₈ alkyl aryl, C₁-C₈ heteroalkyl, C₁-C₈ heterocycloalkyl, heteroaryl, and NHCR_{A}R_{A′}C(O)R₁₂.

In one particularly preferred variant of the first embodiment, the compound of the invention is selected from among the compounds having the formula I-A to I-J:

TABLE 1

| Compounds | |
|---|---|
| (anomers) | Structure |
| I-A (beta) | 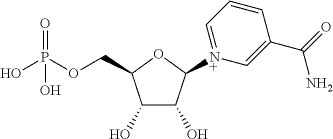 [SMILES: NC(=O)c1ccc[n+]([C@@H]2O[C@H](COP(=O)(O)O)[C@@H](O)[C@H]2O)c1] |
| I-B (alpha) | 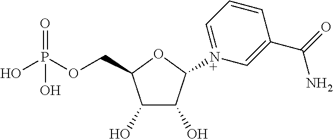 [SMILES: NC(=O)c1ccc[n+]([C@H]2O[C@H](COP(=O)(O)O)[C@@H](O)[C@H]2O)c1] |
| I-C (beta) | 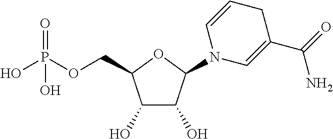 [SMILES: NC(=O)C1=CN([C@@H]2O[C@H](COP(=O)(O)O)[C@@H](O)[C@H]2O)C=CC1] |
| I-D (alpha) | 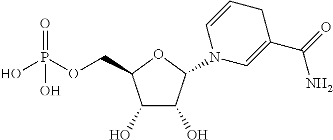 [SMILES: NC(=O)C1=CN([C@H]2O[C@H](COP(=O)(O)O)[C@@H](O)[C@H]2O)C=CC1] |
| I-E (beta) | 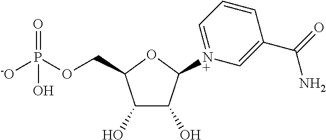 |
| I-F (alpha) | 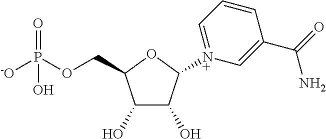 |
| I-G (beta) | 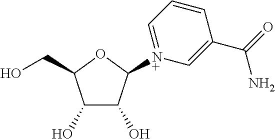 [SMILES: NC(=O)c1ccc[n+]([C@@H]2O[C@H](CO)[C@@H](O)[C@H]2O)c1] |
| I-H (alpha) | 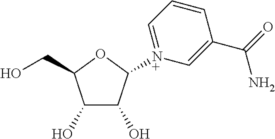 [SMILES: NC(=O)c1ccc[n+]([C@H]2O[C@H](CO)[C@@H](O)[C@H]2O)c1] |
| I-I (beta) | 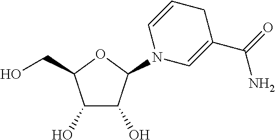 [SMILES: NC(=O)C1=CN([C@@H]2O[C@H](CO)[C@@H](O)[C@H]2O)C=CC1] |
| I-J (alpha) | 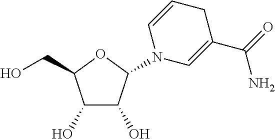 [SMILES: NC(=O)C1=CN([C@H]2O[C@H](CO)[C@@H](O)[C@H]2O)C=CC1] |

In a preferred second embodiment, the derivative is the compound having the formula (I a).

In one variant of the second embodiment, X′1 and X′2 each independently represent an oxygen.

In one variant of the second embodiment, R′7 and R′14 each independently represent an NH₂.

In one variant of the second embodiment, R′1 and/or R′13 each independently represent a hydrogen.

In one variant of the second embodiment, R′6 and/or R′8 each independently represent a hydrogen.

In one variant of the second embodiment, R′2, R′3, R′4, R′5, R′9, R′10, R′11, and R′12 each independently represent a hydrogen.

In one variant of the second embodiment, R′2, R′3, R′4, R′5, R′9, R′10, R′11, and R′12 each independently represent an OH.

In one variant of the second embodiment, Y′1 and Y′2 each independently represent a CH.

In one variant of the second embodiment, Y′1 and Y′2 each independently represent a CH2.

In one variant of the second embodiment, the compound according to the invention is selected from among the compounds having the formula Ia-A to Ia-I:

TABLE 2

| Compound | Structure |
|---|---|
| Ia-A (beta, beta) | 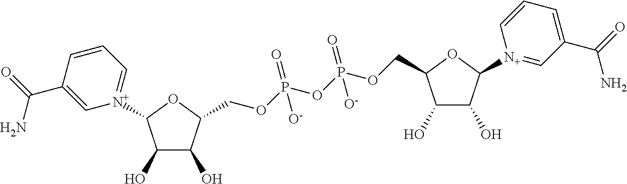 [SMILES: NC(=O)c1ccc[n+]([C@@H]2O[C@H](COP(=O)([O-])OP(=O)([O-])OC[C@H]3O[C@@H]([n+]4cccc(C(N)=O)c4)[C@H](O)[C@@H]3O)[C@@H](O)[C@H]2O)c1] |
| Ia-B (beta, alpha) | 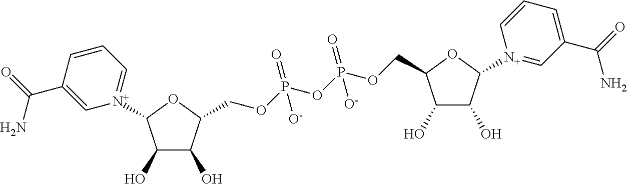 [SMILES: NC(=O)c1ccc[n+]([C@@H]2O[C@H](COP(=O)([O-])OP(=O)([O-])OC[C@H]3O[C@H]([n+]4cccc(C(N)=O)c4)[C@H](O)[C@@H]3O)[C@@H](O)[C@H]2O)c1] |
| Ia-C (alpha, alpha) | 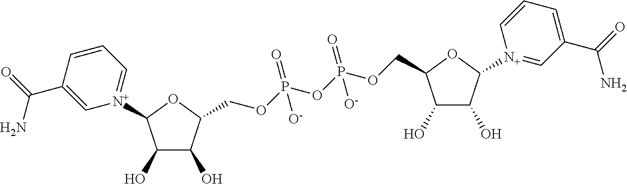 [SMILES: NC(=O)c1ccc[n+]([C@H]2O[C@H](COP(=O)([O-])OP(=O)([O-])OC[C@H]3O[C@H]([n+]4cccc(C(N)=O)c4)[C@H](O)[C@@H]3O)[C@@H](O)[C@H]2O)c1] |
| Ia-D (beta, beta) | 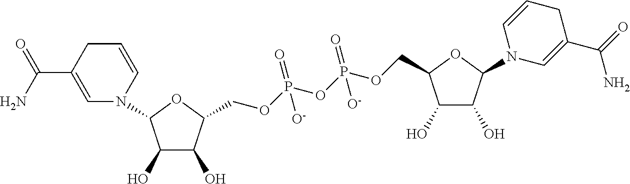 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 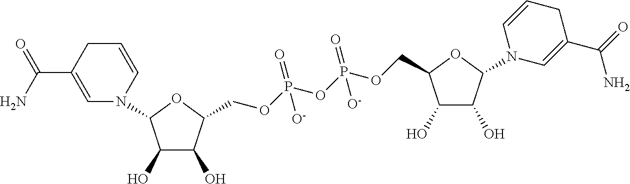 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 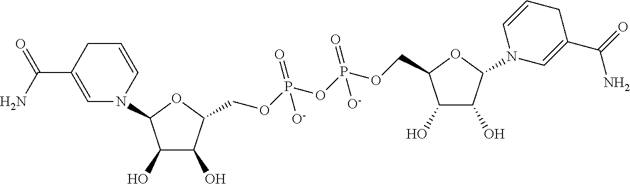 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 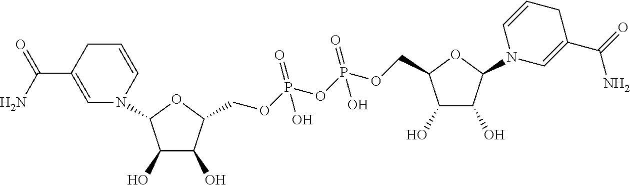 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 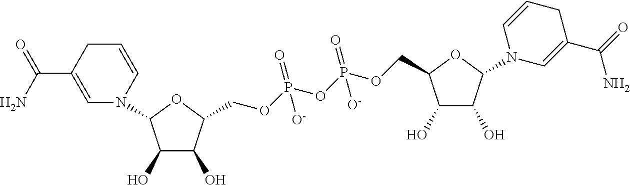 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 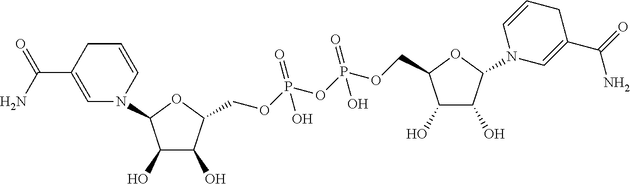 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

In one particularly preferred variant of the first embodiment, the derivative is alpha-NMN having the formula:

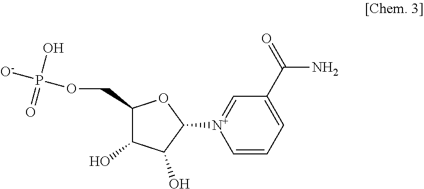 [SMILES: NC(=O)c1ccc[n+]([C@H]2O[C@H](COP(=O)([O-])O)[C@@H](O)[C@H]2O)c1]

In a preferred fourth embodiment, the derivative is NMN-H:

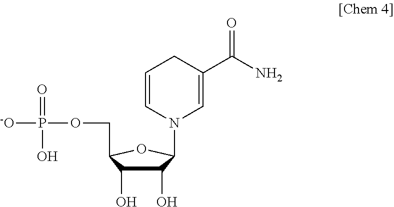

Advantageously, the precursor is nicotinamide riboside:

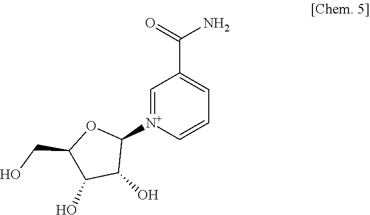 [SMILES: NC(=O)c1ccc[n+]([C@@H]2O[C@H](CO)[C@@H](O)[C@H]2O)c1]

or dihydronicotinamide riboside (denoted —NR—H) of formula:

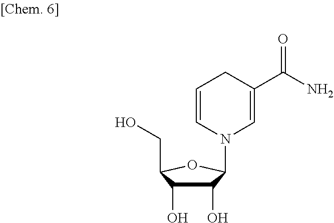

Preferably, the composition of the invention is not a therapeutic composition.

Advantageously, the composition of the invention can treat the signs of ageing of dry skin.

Advantageously, the composition of the invention can treat the signs of ageing of normal or mixed skin.

Advantageously, the composition of the invention can treat the signs of ageing of oily skin.

Advantageously, the composition of the invention can treat the signs of ageing of normal skin.

Advantageously, the composition of the invention can reduce swelling of the lower eyelid and/or can reduce dark circles.

The composition of the invention can reduce wrinkles and/or can increase firmness of the skin and/or can increase the elasticity of dry skin, of normal skin and of mixed skin.

In one embodiment, the composition of the invention can reduce the roughness of the epidermis of dry skin and/or increase the smooth appearance of the epidermis of dry skin and/or increase the thickness of the epidermis in particular of dry skin.

Advantageously, the composition of the invention may comprise between 0.01 weight % and 30 weight %, preferably between 0.1% and 10 weight %, more preferably between 1% and 5 weight %, and further preferably about 2 weight % of NMN, a derivative thereof, a precursor thereof, or a salt thereof, relative to the total weight of the composition.

In one preferred embodiment, the composition of the invention comprises an extract of Edelweiss.

Advantageously, the composition of the invention may comprise between 0.001 weight % and 10 weight %, preferably between 0.005% and 5 weight %, more preferably between 0.01% and 1 weight %, further preferably about 0.04 weight % of extract of Edelweiss, relative to the total weight of the composition.

In one preferred embodiment, the composition of the invention may further comprise hyaluronic acid.

Advantageously, the composition of the invention may comprise between 0.01% and 5% of hyaluronic acid, preferably between 0.05% and 2% of hyaluronic acid, more preferably between 0.1% and 1% of hyaluronic acid, and most preferably about 0.35% of hyaluronic acid by weight of the composition.

Preferably, the hyaluronic acid is contained in the form of a sodium salt of hyaluronic acid.

Advantageously, the composition of the invention may further comprise at least one cosmetically acceptable adjuvant selected from the group formed by thickeners, humectants, emollients, surfactants, preserving agents, anti-foaming agents, pH adjusters, gelling agents, diluents, waxes, vegetable oils, animal oils, butters, emulsifiers, vitamins, vitamin precursors, natural plant extracts, active peptides, anti-pigment active agents and/or colouring agents and combinations thereof.

Advantageously, the composition may further comprise at least one ingredient selected from among beech bud extract, green algae extract, magnolia bark extract, grape seed extract and combinations thereof.

In one embodiment, the composition comprises beech bud extract and green algae extract.

In one embodiment, the composition comprises magnolia bark extract and grape seed extract.

In one preferred embodiment, the composition of the invention may further comprise hyaluronic acid, glycolic acid, fruit acids, anti-UVA filters, anti-UVB filters, vitamins, vitamin precursors, natural plant extracts, active peptides and combinations thereof.

Advantageously, the composition of the invention can be in the form of a gel, solution, water-in-oil emulsion, oil-in-water emulsion, an oil, cream, ointment, liposomal dispersion, or a liniment.

Advantageously, the composition of the invention can be intended to be applied to the skin of the face and/or body, preferably the face.

Advantageously, the composition of the invention can be a serum, universal cream, day cream, night cream, eye contour cream, makeup removing lotion, makeup removing milk, makeup removing oil, makeup removing balm, cosmetic mask, toning lotion, exfoliating product, cleansing gel, cleansing foam, cleansing milk, cleansing lotion.

In one preferred embodiment, the composition of the invention is a serum.

In one variant of this embodiment, the composition of the invention is a serum for the face.

In one variant of this embodiment, the composition of the invention is a serum for the eye contour.

Advantageously, the pH of the composition of the invention can lie between 4 and 9, preferably between 5 and 7.

A further subject of the present invention is a non-therapeutic cosmetic method for treating signs of ageing, comprising the application to the skin surface of a composition of the invention such as defined above.

A further subject of the invention is the cosmetic use of nicotinamide mononucleotide (NMN), a derivative thereof, a precursor thereof or a salt thereof, or combinations thereof to prevent or treat at least one sign of ageing such as defined above.

### Definitions

In the present invention, the following terms have the following meaning.

Unless otherwise indicated, the nomenclature of substituents which are not explicitly defined in the present invention is obtained by naming the terminal portion of the functional group followed by the adjacent functional group towards the point of attachment.

«Alkyl» on its own or as part of another substituent refers to a hydrocarbyl radical having the formula CnH2n+1 in which n is a number greater than or equal to 1. In general, the alkyl groups of this invention include from 1 to 12 carbon atoms, preferably from 1 to 8 carbon atoms, more preferably from 1 to 6 carbon atoms, even more preferably from 1 to 2 carbon atoms. The alkyl groups may be linear or branched and may be substituted as indicated in the present invention. The alkyls that are suitable for the purposes of implementation of the invention may be selected from among methyl, ethyl, n-propyl, i-propyl, n-butyl, i-butyl, s-butyl and t-butyl; pentyl and its isomers such as n-pentyl and iso-pentyl; and hexyl and its isomers such as n-hexyl and iso-hexyl; heptyl and its isomers (for example n-heptyl, iso-heptyl); octyl and its isomers (for example n-octyl, iso-octyl); nonyl and its isomers (for example n-nonyl, iso-nonyl); decyl and its isomers (for example n-decyl, iso-decyl); undecyl and its isomers; dodecyl and its isomers. Preferably, the alkyl groups may be selected from among methyl, ethyl, n-propyl, i-propyl, n-butyl, i-butyl, s-butyl, t-butyl, n-pentyl, n-hexyl, n-heptyl, n-octyl, n-nonyl, and n-decyl. The saturated and branched alkyl groups may be selected, without limitation, from among isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, and 3,3-diethylhexyl. The preferred alkyl groups are the following: methyl, ethyl, n-propyl, i-propyl, n-butyl, i-butyl, s-butyl and t-butyl. Cx-Cy-alkyls refer to alkyl groups that comprise from x to y carbon atoms.

When the suffix “ene” (“alkylene”) is used in conjunction with an alkyl group, it indicates that the alkyl group as defined herein has two single bonds as points of attachment to other groups. The term “alkylene” includes methylene, ethylene, methylmethylene, propylene, ethylethylene, and 1,2-diméthylethylene.

The term “alkenyl” as used herein refers to an unsaturated hydrocarbyl group, which may be linear or branched, that comprises one or more carbon-carbon double bonds. The alkenyl groups that are suitable comprise between 2 and 12 carbon atoms, preferably between 2 and 8 carbon atoms, and even more preferably between 2 and 6 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and other similar groups.

The term “alkynyl”, as used herein, refers to a class of monovalent unsaturated hydrocarbyl groups, in which the unsaturation results from the presence of one or more carbon-carbon triple bond(s). The alkynyl groups generally, and preferably, have the same number of carbon atoms as described here above for the alkenyl groups. Without limitation, some examples of alkynyl groups include ethynyl, 2-propynyl, 2-butynyl, 3-butynyl, 2-pentynyl and its isomers, 2-hexynyl and the isomers thereof, etc.

«Alkoxy» refers to an alkyl group as defined here above, which is attached to another moiety by means of an oxygen atom. Examples of alkoxy groups include the groups: methoxy, isopropoxy, ethoxy, tert-butoxy, and the like. The alkoxy groups may be optionally substituted by one or more substituent(s). The alkoxy groups included in the compounds of this invention may be optionally substituted with a solubilizing group.

«Aryl», as used herein, refers to a polyunsaturated aromatic hydrocarbyl group having a single ring (for example phenyl) or multiple aromatic rings that are fused together (for example naphthyl) or covalently bonded, which generally contains 5 to 18 atoms, preferably 5 to 12, on a more preferred basis 6 to 10, with at least one of the said rings being aromatic. The aromatic ring may optionally include one or two additional rings (cycloalkyl, heterocyclyl, or heteroaryl) fused thereto. The aryl is also intended to include partially hydrogenated derivatives of the carbocyclic systems listed herein. Examples of aryl include phenyl, biphenylyl, biphenylenyl, 5- or 6-tetralinyl; naphthalene-1- or -2-yl; 4-, 5-, 6 or 7-indenyl; 1-, 2-, 3-, 4-, or 5-acenaphthylenyl; 3-, 4-, or 5-acenaphthenyl; 1-, or 2-pentalenyl; 4-, or 5-indanyl; 5-, 6-, 7-, or 8-tetrahydronaphthyl; 1,2,3,4-tetrahydronaphthyl; 1,4-dihydronaphthyl; 1-, 2-, 3-, 4-, or 5-pyrenyl.

When at least one carbon atom in an aryl group is replaced by a heteroatom, the resulting ring is referred to herein as a “heteroaryl” ring.

«Alkylaryl» designates an aryl group substituted by an alkyl group.

«Amino acid» refers to an alpha-amino carboxylic acid, that is to say, a molecule comprising a carboxylic acid functional group and an amino functional group in the alpha position of the carboxylic acid group, for example a proteinogenic amino acid or a non-proteinogenic amino acid.

«Proteinogenic amino acid» refers to an amino acid that is incorporated into the proteins during the translation of the messenger RNA by the ribosomes in living organisms, that is to say, Alanine (ALA), Arginine (ARG), Asparagine (ASN), Aspartate (ASP), Cysteine (CYS), Glutamate (glutamic acid) (GLU), Glutamine (GLN), Glycine (GLY), Histidine (HIS), Isoleucine (ILE), Leucine (LEU), Lysine (LYS), Methionine (MET), Phenylalanine (PHE), Proline (PRO), Pyrrolysine (PYL), Selenocysteine (SEL), Serine (SER), Threonine (THR), Tryptophan (TRP), Tyrosine (TYR), or Valine (VAL).

«Non-proteinogenic amino acid» as used herein refers to an amino acid that is not naturally encoded or found in the genetic code of a living organism. Without limitation, some examples of non-proteinogenic amino acid are: ornithine, citrulline, argininosuccinate, homoserine, homocysteine, cysteine-sulfinic acid, 2-aminomuconic acid, δ-aminolevulinic acid, β-alanine, cystathionine, γ-aminobutyrate, dihydroxyphenylalanine (DOPA), 5-hydroxytryptophan, D-serine, ibotenic acid, α-aminobutyrate, 2-aminoisobutyrate, D-leucine, D-valine, D-alanine, or D-glutamate

The term “cycloalkyl” as used herein refers to a cyclic alkyl group, i.e. a monovalent, saturated or unsaturated hydrocarbyl group, having 1 or 2 ring structures. The term “cycloalkyl” includes monocyclic or bicyclic hydrocarbyl groups. The cycloalkyl groups may comprise 3 or more carbon atom(s) in the ring and generally, according to the present invention, comprise from 3 to 10, more preferably from 3 to 8 carbon atoms, and even more preferably from 3 to 6 carbon atoms. Examples of cycloalkyl groups include, but are not limited to, cyclopropyl, cyclobutyl, cyclopentyl, cyclohexyl, with cyclopropyl being particularly preferred.

«Halogen» or «halo» means fluoro, chloro, bromo or iodo. The preferred halo groups are fluoro and chloro.

«Haloalkyl» alone or in combination, designates an alkyl radical having the meaning such as defined above, wherein one or more hydrogen atoms are replaced by a halogen such as defined above. Among the examples of said halogenoalkyl radicals, mention can be made of chloromethyl, 1-bromoethyl, fluoromethyl, difluoromethyl, trifluoromethyl, 1,1,1-trifluoroethyl and similar radicals. Cx-Cy-haloalkyl and Cx-Cy-alkyl refer to alkyl groups that comprise from x to y carbon atoms. The preferred haloalkyl groups are difluoromethyl and trifluoromethyl.

«Heteroalkyl» refers to an alkyl group as defined here above, in which one or more carbon atom(s) are replaced by a heteroatom selected from among oxygen, nitrogen and sulfur atoms. In the heteroalkyl groups, the heteroatoms are bonded along the alkyl chain only to carbon atoms, that is to say, each heteroatom is separated from every other heteroatom by at least one carbon atom. However, the nitrogen and sulfur heteroatoms may optionally be oxidised and the nitrogen heteroatoms may optionally be quaternised. A heteroalkyl is bonded to another group or molecule only by means of a carbon atom, that is to say, the bonding atom is not selected from the heteroatoms included in the heteroalkyl group.

The term “heteroaryl” as used herein, alone or as part of another group, refers to, but is not limited to, aromatic rings of 5 to 12 carbon atoms or ring systems containing 1 or 2 rings that are fused or covalently bonded, and generally containing 5 or 6 atoms, with at least one of the said rings being aromatic; in which one or more carbon atom(s) in one or more of these rings are replaced by oxygen, nitrogen and/or sulfur atoms, it being possible for the nitrogen and sulfur heteroatoms to optionally be oxidised and for the nitrogen heteroatoms to optionally be quaternised. These rings may be fused to an aryl, cycloalkyl, heteroaryl or heterocyclyl ring. Without limitation, some examples of such heteroaryls include: furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, dioxinyl, thiazinyl, triazinyl, imidazo [2,1-b] [1,3] thiazolyl, thieno [3,2-b] furanyl, thieno [3,2-b] thiophenyl, thieno [2,3-d] [1,3] thiazolyl, thieno [2,3-d] imidazolyl, tetrazolo [1,5-a] pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo [l,2-a] pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

When at least one carbon atom in a cycloalkyl group is replaced by a heteroatom, the resulting ring is referred to herein as “heterocycloalkyl” or “heterocyclyl”.

The terms “heterocyclyl”, “heterocycloalkyl” or “heterocyclo”, as used herein on their own or as part of another group, refer to non-aromatic cyclic groups, either fully saturated or partially unsaturated (for example, 3 to 7 membered monocyclic, 7 to 11 membered bicyclic groups or containing a total of 3 to 10 ring atoms), which have at least one heteroatom in at least one ring containing a carbon atom. Each ring of the heterocyclic group containing a heteroatom may have 1, 2, 3, or 4 heteroatoms selected from among nitrogen, oxygen and/or sulfur atoms, where the nitrogen and sulfur heteroatoms may optionally be oxidised, and the nitrogen heteroatoms may optionally be quaternised.

Any whichever of the carbon atoms of the heterocyclic group may be substituted by an oxo (for example piperidone, pyrrolidinone). The heterocyclic group may be attached to any heteroatom or carbon atom in the ring or ring system, where the valence so permits. The rings of multi-ring heterocycles may be fused, bridged and/or connected by one or more spiro atoms. Exemplary heterocyclic groups include, but are not limited to, the following groups: oxetanyl, piperidinyl, azetidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholine-4-yl, thiomorpholine-4-ylsulf oxide, thiomorpholine-4-ylsulfone, 1,3-dioxolanyl, 1,4-oxathianyl, 1H-pyrrolizinyl, tetrahydro-1,1-dioxothiophenyl, N-formylpiperazinyl, and morpholine-4-yl.

The term “precursor” as used herein also refers to pharmacologically acceptable derivatives of compounds having the formula (I) or (Ia) such as esters, of which the in vivo biotransformation product is the active drug. Precursors are characterised by increased bioavailability and are readily metabolised into active compounds in vivo. The precursors that are appropriate for the purposes of the invention include in particular carboxylic esters, in particular alkyl esters, aryl esters, acyloxyalkyl esters, and the carboxylic esters of dioxolene; ascorbic acid esters.

The term “active ingredient” refers to a molecule or a substance which, when administered to a subject, slows down or stops the progression, aggravation or deterioration of one or more symptoms of a condition; relieves the symptoms of a condition; cures a condition.

The «salts» comprise the addition salts of acids and bases of these salts. The suitable acid addition salts are formed from acids which form non-toxic salts. For example, these are the salts acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, l′hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, and xinofoate salts. The suitable basic salts are formed from bases which form non-toxic salts. As examples, mention can be made of the salts of aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine and zinc. Hemi-salts of acids and bases can also be formed, for example hemisulfates and salts of chemical calcium. The preferred salts are hydrochloride/chloride, bromide/hydrobromide, bisulfate/sulfate, nitrate, citrate and acetate.

The acceptable salts can be prepared with one of more of the following methods:

by reacting the compound with the desired acid;

by reacting the compound with the desired base;

by removing a labile protecting group in an acid or basic medium from a suitable precursor of the compound, or by ring opening of a suitable cyclic precursor, for example a lactone or a lactam, using the desired acid; or

by converting one salt of the compound into another by reacting the initial salt with an appropriate acid or by means of an appropriate ion exchange column.

All of these reactions are generally carried out in solution. The salt can precipitate out of the solution and may be collected by filtration or may be recovered by evaporation of the solvent. The degree of ionisation of the salt may vary from completely ionised to almost non-ionised.

The term «solvate» is used herein to describe a molecular complex comprising the compound of the invention and one or more acceptable solvent molecules, for example ethanol.

The term “substituent” or “substituted” indicates that a hydrogen radical on a compound or a group is replaced by any desired group which is substantially stable under the reaction conditions in an unprotected form or when it is protected by a protecting group. Examples of preferred substituents include, but are not limited to: a halogen (chloro, iodo, bromo, or fluoro); an alkyl; an alkenyl; an alkynyl, as described here above; a hydroxy; an alkoxy; a nitro; a thiol; a thioether; an imine; a cyano; an amido; a phosphonato; a phosphine; a carboxyl; a thiocarbonyl; a sulfonyl; a sulfonamide; a ketone; an aldehyde; an ester; an oxygen (—O); a haloalkyl (for example, trifluoromethyl); a cycloalkyl, which may be condensed-ring or non-condensed-ring monocyclic or polycyclic (for example, cyclopropyl, cyclobutyl, cyclopentyl or cyclohexyl); or a heterocycloalkyl, which may be condensed-ring or non-condensed-ring monocyclic or polycyclic (for example, pyrrolidinyl, piperidinyl, piperazinyl, morpholinyl, or thiazinyl); fused or unfused monocyclic or polycyclic, aryl or heteroaryl (for example, aryl, heteroaryl, pyrrolidinyl, piperidinyl, piperazinyl, morpholinyl, or thiazinyl); fused or unfused monocyclic or polycyclic (for example, aryl, heteroaryl, pyrrolidinyl, piperidinyl, piperazinyl, morpholinyl, or thiazinyl), phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazidinyl, pyridaziminyl, pyridaziminyl, benzimidazolyl, benzothiophenyl, or benzofuranyl); amino (primary, secondary or tertiary); CO₂CH₃; CONH₂; OCH₂CONH₂; NH₂; SO₂NH₂; OCHF₂; FC₃; OCF₃; moreover these groups may also be optionally substituted by a fused ring bridge or structure, for example —OCH2O—. These substituents may optionally be further substituted by a substituent selected from among these groups. In certain representations, the term “substituent” or the adjective “substituted” refers to a substituent selected from the group constituted of: an alkyl, an alkenyl, an alkynyl, a cycloalkyl, a cycloalkenyl, a heterocycloalkyl, an aryl, a heteroaryl, an arylalkyl, a heteroarylalkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR₁₃, —NR₁₃S(O)rR₁₄, —OS(O)rR₁₄, S(O)rNR₁₁R₁₂, —O, —S, and —NR₁₃, where r is 1 or 2; R₁₁ and R₁₂, for each occurrence, are independently H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl; or R₁₁ and R₁₂ taken together with the nitrogen to which they are attached are an optionally substituted heterocycloalkyl, or an optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl. In some embodiments; the term “substituent” or the adjective “substituted” refers to a solubilizing group.

The term «administering», or a variant of this term (for example, «to administer»), refers to providing of the active ingredient, whether alone or as part of a cosmetic composition, to the subject in whom/to whom the skin condition is to be treated or prevented.

The terms «to treat», and «treatment», such as used in the present invention are meant to include the relieving, alleviation, or ablation of a condition and/or the symptoms associated therewith.

The terms «prevent», «avoid» and «prevention», as used in the present invention, refer to a method that serves the purpose of delaying or preventing the onset of a condition and/or the symptoms associated therewith, preventing a subject from contracting a condition or reducing the risk of a subject contracting a condition.

By «cosmetic composition» it is meant any mixture intended to be placed in contact with the surface parts of the human body (epidermis, hair, nails, lips and external genital parts), or with teeth and oral mucosa for the sole or chief purpose of cleansing, fragrancing thereof, modifying the appearance thereof, giving protection thereto, maintaining the good condition thereof or for correcting body odour.

By cosmetic «substance» it is meant a chemical element and compounds thereof in the natural state or obtained by a manufacturing process, including any additive required to preserve the stability thereof and any impurity resulting from the process applied, but with the exclusion of any solvent which can be separated without affecting the stability of the substance or modifying the composition thereof.

By «mixture», it is meant a mixture or a solution composed of two or more substances.

By «dark circle», in anatomy called periorbital hyperchromia, it is meant a variation in the colouring of the skin under the eye. Dark circles impart a tired, aged appearance.

The bonds of an asymmetric carbon can be represented herein using a solid triangle ( [symbol] ), a dotted triangle ( [symbol] ) or a zig-zag line ( [symbol] ).

### DETAILED DESCRIPTION OF THE INVENTION

The subject of the present invention is a cosmetic composition comprising nicotinamide mononucleotide (NMN), a derivative thereof, a precursor thereof or a salt thereof, to prevent or treat of at least one sign of ageing.

Nicotinamide adenine dinucleotide (NAD) is a coenzyme present in all living cells. NAD exists in the cell either in its oxidised form NAD+, or in its reduced form NADH. The role of NAD is that of an electron carrier that is involved in redox reactions of the metabolism. NAD is moreover also involved in a number of cellular processes such as adenosine diphosphate (ADP) ribosylation in the context of post-translational modifications of proteins.

NAD can be synthesised de novo by the cell from amino acids such as tryptophan or aspartate. However, such synthesis is marginal because the main pathway for NAD synthesis is the salvage pathway, by means of which the cell, and primarily the cell nucleus, recycles compounds in order to reform NAD from precursors. The precursors of NAD include niacin, nicotinamide riboside, nicotinamide mononucleotide, and nicotinamide.

NMN is one of the compounds that enable the synthesis of NAD by the salvage pathway and has the formula:

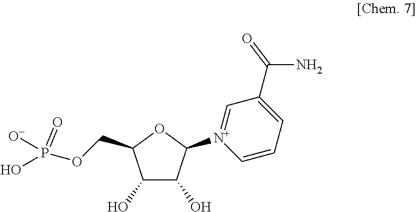 [SMILES: NC(=O)c1ccc[n+]([C@@H]2O[C@H](COP(=O)([O-])O)[C@@H](O)[C@H]2O)c1]

In the meaning of the invention, the derivatives, precursors and salts of NMN are cosmetically acceptable, i.e. they are authorised or at least the use thereof is not prohibited or restricted by European Regulation No 1223/2009 or by the Food and Drug Administration—FDA.

The salts suitable for implementing the invention are non-toxic or slightly toxic and are obtained from organic or non-organic bases or acids of NAD precursors. As examples of salts, mention can be made of chlorides, bromides, fluorides, iodides, sulfates, sodium, potassium, magnesium, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfonate, trifluoromethanesulfonate, trichloromethanesulfonate, tribromomethanesulfonate, and trifluoroacetate. Preferably the salt is a chloride.

In one preferred embodiment, the NMN is in the form of a zwitterion. By «zwitterion» it is meant a molecular chemical species having opposed electrical charges and generally located on non-adjacent atoms of the molecule.

The composition of the invention is particularly effective for the treatment and prevention of signs of ageing. The composition of the invention exhibits good moisturizing and anti-age properties, and it is well tolerated by all skin types.

NMN Derivatives and Precursors

In the invention, the NMN derivative can be selected from among alpha nicotinamide mononucleotide (α-NMN), dihydronicotinamide mononucleotide (denoted NMN-H), the compound of formula (I):

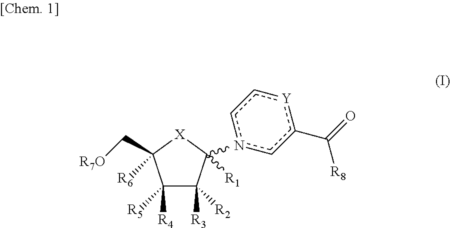

or one of the pharmaceutically acceptable stereoisomers, salts, hydrates, solvates, or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are each independently selected from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, C₁-C₈ alkyl, C₁-C₈ thio-alkyl, C₁-C₈ heteroalkyl and OR; wherein R is selected from among H and C₁-C₈ alkyl;
R₇ is selected from among H, P(O)R₉R₁₀, P(S)R₉R₁₀ and

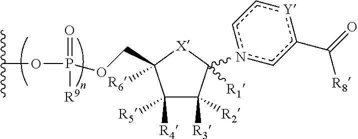

wherein n is an integer equal to 1 or 3; wherein
R₉ and R₁₀ are each independently selected from among OH, OR₁₁, NHR₁₃, NR₁₃R₁₄, a (C₁-C₈) alkyl, a (C₂-C₈) alkenyl, a (C₂-C₈) alkynyl, a (C₃-C₁₀) cycloalkyl, a (C₅-C₁₂) aryl, (C₁-C₈) alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from among 1 to 8; and P(O)(OH)OP(O)(OH)₂, halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂ SO² in which each of R₁₁ₐ is independently selected from among H and (C₁-C₆) alkyl and R_{11b} is independently selected from among OH, C₁-C₆ alkoxy, NH₂, NH(C₁-C₆ alkyl) or N(C₁-C₆ alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted by one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀, together with the phosphorus atoms to which they are attached, form a 6-membered ring in which —R₉-R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
   R₉ and R₁₀, together with the phosphorus atoms to which they are attached, form a 6-membered ring in which —R₉-R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
Rₐ is selected from among H, OR, NHR₁₃, NR₁₃R₁₄, NH—NHR₁₃, SH, CN, N₃ and halogen; where R₁₃ and R₁₄ are each independently selected from among H, (C₁-C₈) alkyl and (C₁-C₈) alkyl aryl; and
R₁₃ and R₁₄ are each independently selected from among H, C₁-C₈ alkyl and C₁-C₈ alkyl aryl;
R₁₅ and R₁₆ are independently selected from among H, C₁-C₈alkyl and C₁-C₈alkyl aryl; and —CR_{B}R_{C}—C(O)—OR_{D} in which R_{B} and R_{C} are independently selected from among H, C₁-C₆ alkyl, C₁-C₆ alkoxy, benzyl, indolyl or imidazolyl, wherein the groups C₁-C₆ alkyl and C₁-C₆ alkoxy can optionally and each independently be substituted by one or more from among a halogen, an amino, amido, guanidyl, hydroxyl, thiol or carboxyl group, and the benzyl group is optionally substituted by one or more from among a halogen or hydroxyl group, or R_{B} and R_{C} together with the carbon atom to which they are attached form a C₃-C₆ cycloalkyl group optionally substituted by one or more from among a halogen, an amino, amido, guanidyl, hydroxyl, thiol or carboxyl group, and R_{D} is H, C₁-C₆ alkyl, C₂-C₆ alkenyl, C₂-C₆ alkynyl or C₃-C₆ cycloalkyl;
Y is selected from among CH, CH₂, C(CH₃)₂ and CCH₃;
[symbol] represents a single or double bond depending on Y; and
[symbol] represents the alpha or beta anomer depending on the position of R₁
   or one of the stereoisomers, salts, hydrates, solvates or crystals thereof
   or
   the compound of formula (Ia):

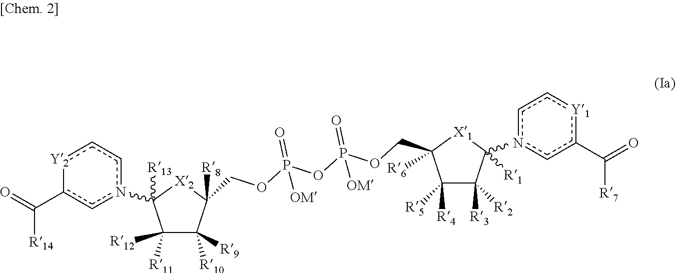

or one of the stereoisomers, salts, hydrates, solvates or crystals thereof, in which:
X′₁ and X′₂ are independently selected from among O, CH₂, S, Se, CHF, CF₂, and C═CH₂;
R′₁ and R′13 are independently selected from among H, azido, cyano, a C₁-C₈ alkyl, a C₁-C₈ thio-alkyl, a C₁-C₈ heteroalkyl, and OR, wherein R is selected from H and a C₁-C₈ alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and a halogen; wherein R and R′ are independently selected from H and a (C₁-C₈) alkyl aryl;
Y′₁ and Y′₂ are independently selected from among CH, CH₂, C(CH₃)₂ or CCH³;
M′ is selected from among H or a suitable counter ion; [symbol]
represents a single or double bond depending on Y′1 and Y′2; and [symbol] represents an alpha or beta anomer depending on the position of R′₁ and R′₁₃;
and combinations thereof.

In a first preferred embodiment, the acceptable derivative is the compound having the formula (I).

In one variant of the first embodiment, X represents an oxygen.

In one variant of the first embodiment, R₁ and R₆ are each independently a hydrogen.

In one variant of the first embodiment, R₂, R₃, R₄ and R₅ are each independently a hydrogen or OH.

In one variant of the first embodiment, Y represents a CH.

In one variant of the first embodiment Y represents a CH₂.

In one variant of the first embodiment, R₇ represents a hydrogen.

In one variant of the first embodiment, R₇ represents P(O)(OH)₂.

In one variant of the first embodiment, the compound of the invention is selected from among the compounds having the formula I-A to I-J:

TABLE 1

| Compounds | |
|---|---|
| (anomers) | Structure |
| I-A (beta) | 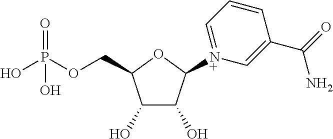 [SMILES: NC(=O)c1ccc[n+]([C@@H]2O[C@H](COP(=O)(O)O)[C@@H](O)[C@H]2O)c1] |
| I-B (alpha) | 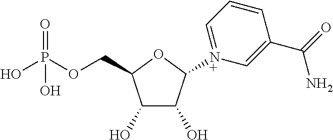 [SMILES: NC(=O)c1ccc[n+]([C@H]2O[C@H](COP(=O)(O)O)[C@@H](O)[C@H]2O)c1] |
| I-C (beta) | 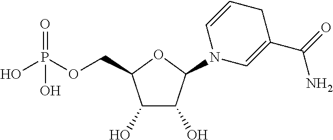 [SMILES: NC(=O)C1=CN([C@@H]2O[C@H](COP(=O)(O)O)[C@@H](O)[C@H]2O)C=CC1] |
| I-D (alpha) | 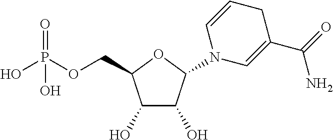 [SMILES: NC(=O)C1=CN([C@H]2O[C@H](COP(=O)(O)O)[C@@H](O)[C@H]2O)C=CC1] |
| I-E (beta) | 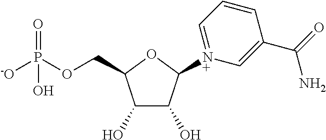 |
| I-F (alpha) | 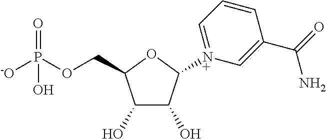 |
| I-G (beta) | 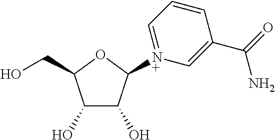 [SMILES: NC(=O)c1ccc[n+]([C@@H]2O[C@H](CO)[C@@H](O)[C@H]2O)c1] |
| I-H (alpha) | 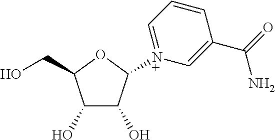 [SMILES: NC(=O)c1ccc[n+]([C@H]2O[C@H](CO)[C@@H](O)[C@H]2O)c1] |
| I-I (beta) | 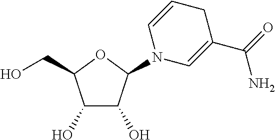 [SMILES: NC(=O)C1=CN([C@@H]2O[C@H](CO)[C@@H](O)[C@H]2O)C=CC1] |
| I-J (alpha) | 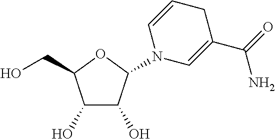 [SMILES: NC(=O)C1=CN([C@H]2O[C@H](CO)[C@@H](O)[C@H]2O)C=CC1] |

In a preferred second embodiment, the acceptable derivative is the compound having the formula (Ia).

In one variant of the second embodiment, X′1 and X′2 each independently represent an oxygen.

In one variant of the second embodiment, R′7 and R′14 each independently represent an NH₂.

In one variant of the second embodiment, R′1 and/or R′13 each independently represent a hydrogen.

In one variant of the second embodiment, R′6 and/or R′8 each independently represent a hydrogen.

In one variant of the second embodiment, R′2, R′3, R′4, R′5, R′9, R′10, R′11 and R′12 each independently represent a hydrogen.

In one variant of the second embodiment, R′2, R′3, R′4, R′5, R′9, R′10, R′11 and R′12 each independently represent an OH.

In one variant of the second embodiment, Y′1 and Y′2 each independently represent a CH.

In one variant of the second embodiment, Y′1 and Y′2 each independently represent a CH2.

In one variant of the second embodiment, the compound of the invention is selected from among the compounds having the formula Ia-A to Ia-I:

TABLE 2

| Compound | Structure |
|---|---|
| Ia-A (beta, beta) | 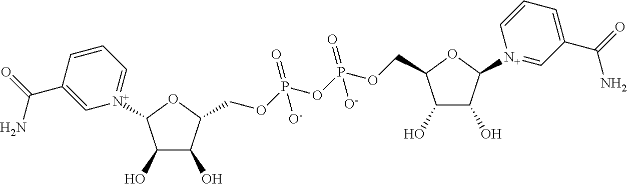 [SMILES: NC(=O)c1ccc[n+]([C@@H]2O[C@H](COP(=O)([O-])OP(=O)([O-])OC[C@H]3O[C@@H]([n+]4cccc(C(N)=O)c4)[C@H](O)[C@@H]3O)[C@@H](O)[C@H]2O)c1] |
| Ia-B (beta, alpha) | 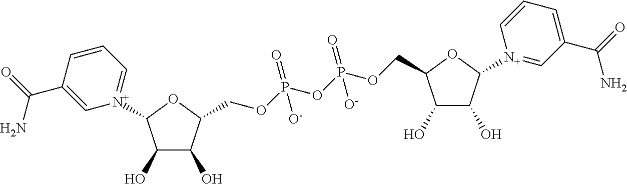 [SMILES: NC(=O)c1ccc[n+]([C@@H]2O[C@H](COP(=O)([O-])OP(=O)([O-])OC[C@H]3O[C@H]([n+]4cccc(C(N)=O)c4)[C@H](O)[C@@H]3O)[C@@H](O)[C@H]2O)c1] |
| Ia-C (alpha, alpha) | 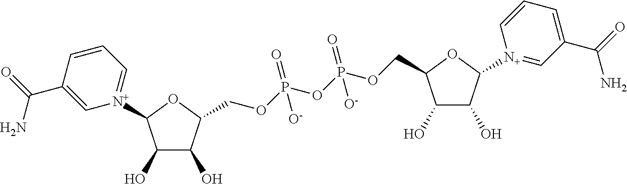 [SMILES: NC(=O)c1ccc[n+]([C@H]2O[C@H](COP(=O)([O-])OP(=O)([O-])OC[C@H]3O[C@H]([n+]4cccc(C(N)=O)c4)[C@H](O)[C@@H]3O)[C@@H](O)[C@H]2O)c1] |
| Ia-D (beta, beta) | 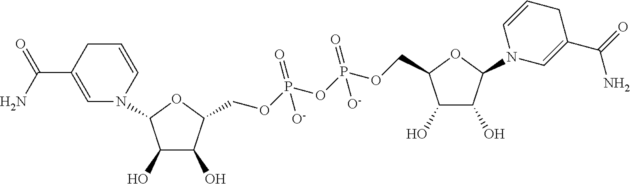 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 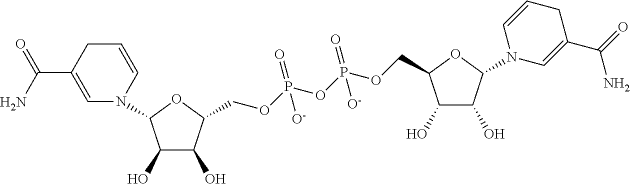 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 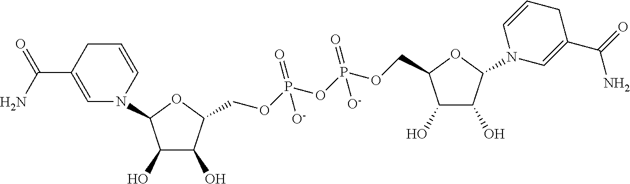 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 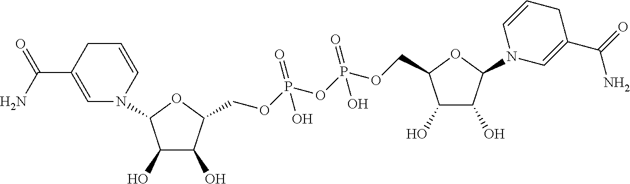 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 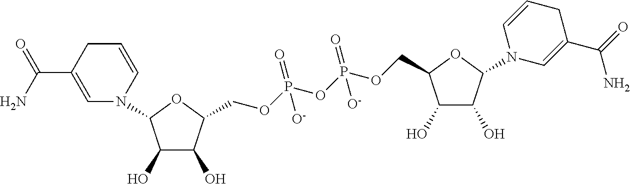 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 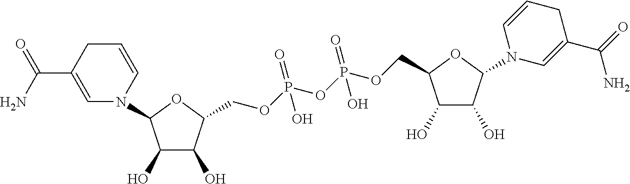 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

In one preferred variant of the first embodiment, the derivative is alpha-NMN having the formula:

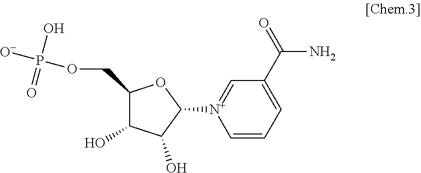 [SMILES: NC(=O)c1ccc[n+]([C@H]2O[C@H](COP(=O)([O-])O)[C@@H](O)[C@H]2O)c1]

In a preferred fourth embodiment, the derivative is NMN-H:

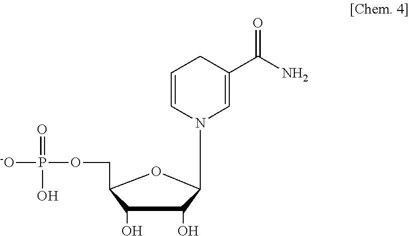

Advantageously the precursor is nicotinamide riboside (denoted NR):

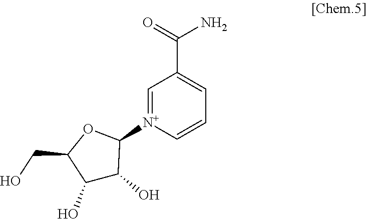 [SMILES: NC(=O)c1ccc[n+]([C@@H]2O[C@H](CO)[C@@H](O)[C@H]2O)c1]

or dihydronicotinamide riboside (denoted —NR—H) of formula:

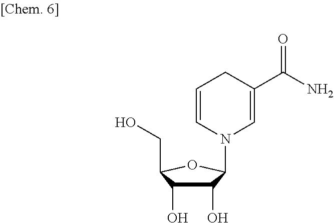

or combination thereof.

Preferably, the derivative of NMN is dihydronicotinamide mononucleotide (NMN-H) and/or alpha-NMN.

Uses

The inventors have shown that the compositions of the invention comprising NMN, a precursor thereof, a derivative thereof and combinations thereof allow the treating of at least one of ageing.

The sign of ageing can be selected from among wrinkles, an increase in skin roughness, reduced thickness of the epidermis or dermis, reduced firmness of the epidermis and dermis, reduced elasticity of the epidermis and dermis, diminished skin radiance, swelling of the lower eyelid, dark circles, erythema of the skin under the eyes, and combinations thereof.

By reducing roughness or increasing the smooth appearance of the skin, it is meant reducing the parameter Ra. The parameter «Ra» corresponds to mean roughness. Ra (in μm) is the arithmetic mean of absolute values of heights, making no distinction between peaks and valleys, over the length of the profile (see FIG. 6):

$$Ra = \frac{1}{l} * {{\int }_{0}}^{lr} \vert R ( x ) \vert dx \qquad [ Math 1 ]$$

This parameter can be measured with any method well known to persons skilled in the art, and in particular by fringe projection using a three-dimensional scanner (see FIG. 5). Briefly, the principle of measurement is based on triangulation, in similar manner to laser profiling, but the whole surface of the sample is acquired in a single operation by projecting a stripe pattern onto the surface, generally at an angle of 30°, and by recording the resulting image with a camera oriented perpendicular to the analysed surface. An algorithm then calculates the measured height deviations.

A reduction in wrinkling can be measured by measuring the parameters obtained by fringe projection with a three-dimensional scanner as mentioned above. The examined parameters on each profile are the following: roughness Ra of which a decease is characterized by a smoothing effect, mean roughness depth Rz and amplitude Rt, the decrease in these values reflecting tensor and anti-wrinkle effects.

The parameter Rz is the mean of 5 maximum values over profile length. A maximum corresponds to the difference between the highest peak and the deepest valley within a sampling length Ir (see FIG. 7). The parameter Rz can be calculated as follows: Rz (μm)=(Rz1+Rz2+Rz3+Rz4+Rz5)/5.

The parameter Rt (μm) is the height difference between the highest peak and the deepest valley within the total sampling length (In) (see FIG. 7).

An increase in the elasticity of the epidermis can be measured with any method well known to skilled persons, and in particular using a Cutometer® MPA580 (Courage & Khazaka). This apparatus measures deformation of a skin area subjected to the stress of mechanical suction and the skin's ability to recover. The Cutometer® is used to measure elasticity and skin firmness. The apparatus measures different parameters, and the combination of these different parameters allows the determining of firmness or elasticity measurements. The principle of the device is based on application of negative pressure onto an area of the skin, which can vary from 20 to 500 mbar. The skin area to be measured is drawn into the standard aperture of the probe having a diameter of 2 mm. The penetration depth of the skin inside the probe is determined by a non-contact optical measuring system composed of a light emitter and receiver. The amount of received light is proportional to the penetration depth of the skin. A spring inside the probe allows a constant pressure to be applied to the skin.

Skin firmness can be measured by the parameter R0 which measures the maximum amplitude of penetration depth of the skin. Parameter RO corresponds to value Uf (see FIG. 9). The elasticity of the skin can be measured with the combination of the three following parameters: parameter R2 representing the biological elasticity of the skin i.e. the ability of the skin to return to its original state and determined by calculating the ratio Ua/Uf; parameter R5 representing net elasticity i.e. the immediate deformation of the skin determined by calculating the ratio Ur/Ue; and parameter R7 representing gross elasticity of the skin i.e. the immediate retraction of the skin calculated using the ratio Ur/Uf. An increase in parameter R0 indicates that the skin is firmer. An increase in parameters R2, R5 and R7 indicates that the skin is more elastic. These parameters are measured with the Cutometer® as defined above.

An increase in reduced density of the epidermis can be measured by ultrasound, in particular using a DermaScan® unit. Scanning is carried out by a probe equipped with a transmitter and receiver of ultrasound waves (transducer). To visualise the different layers of the skin (epidermis, dermis, hypodermis), it is possible to use probes having a high frequency (10 to 50 MHz), preferably 20 MHz. This technique allows two-dimensional viewing of the skin to a depth of 13 mm. Measurement of skin thickness is obtained with the ratio between the surface area of non-echogenic tissue and the total measured surface area.

A reduction in the swelling of the lower eyelid, of dark circles under the eye and/or of wrinkles can be measured by a clinical scoring method conducted by a trained clinician or self-evaluation by the subject. For this technique, also known as the Bazin grading scale, photographs are shown to the assessor or to the subject showing different stages of wrinkles and fine lines around the eye (see FIG. 8), of swelling of the lower eyelid (see FIG. 10) and of dark circles (see FIG. 11). Each photograph is associated with a numerical value. The subject chooses the numerical value corresponding to own assessment of the appearance of the sign of ageing under consideration.

Skin radiance can be measured by self-evaluation or using a Glossymeter (Courage & Khazaka). The Glossymeter GL200® (Courage & Khazaka) allows measurement of the radiance (or gloss) of the surface measured by quantification of reflected light.

As shown in FIG. 12, when the probe comes into contact with the measurement area, a white light is projected at an angle of 0° and is reflected by mirrors at an angle of 60° (arrow 1). Part of this light is directly reflected (gloss) at the same angle of 60° (arrow 2=angle of reflection). Another part of this light is absorbed by the surface of the sample and then dispersed and reflected as scattered light (arrows 3). The scattered reflected light is measured at a 0° (i.e. perpendicular to the measured surface). The probe, of 2.5×5 mm, is particularly adapted for measurements on small surface areas. Unlike other devices for measuring gloss, which are only able to compare surfaces that have an almost similar structure, colour and radiance, the Glossymeter GL200® is equipped with Diffuse Scattering Correction (DSC) which reduces the influence of scattered reflection affected by the colour of the area to be measured and allows the comparison of different types of skin/hair/nails/lips. The values are expressed as a «Glossymeter unit», a measurement unit correlated with standard industrial units (GU: gloss unit) based on DIN and ISO standards.

The composition of the invention allows the obtaining of at least one effect from among an increase in hydration, reduced wrinkles, reduced skin roughness, an increase in firmness of the epidermis, increased elasticity of the epidermis, an increase in skin radiance, reduced swelling of the lower eyelid and reduced colouring of dark circles, a decrease in erythema around the eyes, and combinations thereof.

In the meaning of the invention, the use of NMN is not a therapeutic use and the composition of the invention is not a therapeutic composition.

Therefore, the cosmetic compositions of the invention allow the prevention or treatment of at least one sign of ageing in a subject, preferably a human. The NMN and the cosmetic compositions of the invention are therefore well tolerated and do not induce or at least reduce the risks of allergies related to the use of anti-age cosmetic ingredients such as retinol for example.

Compositions

The composition of invention may comprise between 0.01 weight % and 30 weight %, preferably between 0.1 weight % and 10 weight %, more preferably between 1 weight % and 5 weight %, and further preferably about 2 weight % of NMN, a derivative thereof, a precursor thereof or a salt thereof, relative to the total weight of the composition.

In one preferred embodiment, the composition of the invention comprises an extract of Edelweiss. Extract of Edelweiss, having the INCI name Leontopodium alpinum Callus Culture Extract, has antioxidant, anti-inflammatory and humectant properties. It therefore allows maintained water content of the skin, wrinkle reduction, an increase in skin elasticity, in skin thickness and dermal density, and increases the barrier properties of the skin by stimulating the genes of keratinisation and the differentiation of corneocytes. The composition of the invention may therefore comprise between 0.001 weight % and 10 weight %, preferably between 0.005 weight % and 5 weight %, more preferably between 0.01 weight % and 1 weight % and further preferably about 0.04 weight % of extract of Edelweiss, relative to the total weight of the composition. Said composition is of particular interest for the formulation of an anti-age cosmetic composition intended to be applied to the eye contour or for a serum.

In another embodiment, the composition of the invention comprises NMN, a cosmetically acceptable derivative thereof, a cosmetically acceptable precursor thereof and hyaluronic acid for the treatment and/or prevention of at least one sign of ageing. Hyaluronic acid (C₁₄H₂₁NO₁₁) is a glycosaminoglycan widely found in conjunctive tissue of the human body, in epithelial and nerve tissue. It is one of the main components of the extracellular matrix. It contributes towards cell multiplication and migration. The hyaluronic acid is preferably in the form of sodium hyaluronate. Being naturally present in the human body, it is a cosmetic ingredient that is well tolerated by all skin types, including atopic skin types. The composition of the invention may comprise between 0.01% and 5% hyaluronic acid, preferably between 0.05% and 2% hyaluronic acid, more preferably between 0.1% and 1% hyaluronic acid, and most preferably about 0.35% hyaluronic by weight of the composition. Preferably the hyaluronic acid is present in the form of a sodium salt of hyaluronic acid. Preferably the hyaluronic acid is of plant origin.

In one variant of this embodiment, the hyaluronic acid has a molecular weight higher than or equal to 100 kDa. The use of a hyaluronic acid having a molecular weight higher than or equal to 100 kD in the composition of the invention allows the obtaining of a humectant film on the surface of the skin and the fading of fine lines and wrinkles.

In another variant of this embodiment, the hyaluronic acid has a molecular weight of between 20 kDa and 50 kDa, limits included. The use of a hyaluronic acid having said molecular weight in the composition of the invention allows better penetration of the hyaluronic acid into the layers of the epidermis, and thereby the obtaining of a repulping effect.

The composition of the invention may further comprise at least one cosmetically acceptable adjuvant selected from the group formed by thickeners, humectants, emollients, surfactants, preserving agents, anti-foaming agents, preserving agents, pH adjusters, gelling agents, diluents, waxes, vegetable oils, animal oils, butters, emulsifiers, vitamins and vitamin precursors, natural plant extracts, active peptides, anti-pigment active substances and/or colouring agents and combinations thereof. Persons skilled in the art are able to choose which cosmetic adjuvants to use for the formulation of the cosmetic composition of the invention.

As thickener, mention can be made of gums such as guar gum, carrageenan gum, acacia gum, agar-agar gum, xanthan gum, and combinations thereof.

As humectant, mention can be made of glycerol, glycerine, urea, amino acids, lactates and lactic acids, butylene glycol, simple sugars such as glucose, hyaluronic acid and combinations thereof. Humectants allow water to be retained inside the skin and hence reduce dehydration of the skin.

As emollients, mention can be made of squalane, vegetable oils, fatty acids and the derivatives thereof, glycerine, glyceryl stearate, soybean oil glycine, lecithin, and combinations thereof. As surfactant, mention can be made of anionic surfactants such as sodium lauryl sulfate, cationic surfactants such as quaternary ammoniums, amphoteric surfactants such as phospholipids and non-ionic surfactants such as the esters of glycol or glycerol, and combinations thereof.

As anti-foaming agent, mention can be made of di- and tri-methicones, siloxanes, polysilicones, isopropyl alcohol and propyl alcohol, and combinations thereof.

As preserving agent, mention can be made of salicylic acid, vitamin E, sodium benzoate, parabens, anti-oxidants, phenoxyethanol, and combinations thereof.

As pH adjusters, mention can be made of calcium carbonate, citric acid, lactic acid, sodium hydroxide, and combinations thereof.

As gelling agents, mention can be made of aloe vera gel, hyaluronic acid and all forms of hydrogels, and combinations thereof.

As diluents, mention can be made of water, oils, C1-C16 alcohols, and combinations thereof.

As waxes, mention can be made of Carnauba wax, beeswax, ozokerite, Candelilla wax and micro-crystalline waxes, or silicone oils i.e. dimethylpolysiloxane, or fatty acids, C12-C18 fatty alcohols, and combinations thereof.

As modified or non-modified vegetable or animal oils, mention can be made of sweet almond oil, avocado oil, calophyllum oil, castor oil, olive oil, lanoline and derivatives thereof, perhydrosqualene, groundnut oil, wheatgerm oil, flax oil, jojoba oil, apricot kernel oil, walnut oil, palm oil, pistachio oil, sesame oil, rapeseed oil, juniper tar oil, corn germ oil, peach kernel oil, poppyseed oil, pine oil, soybean oil, safflower oil, copra oil, hazelnut oil, grapeseed oil, or sunflower seed oil, esters of saturated or unsaturated fatty acids, or synthetic oils such as ethyl or isopropyl palmitate, alkyl myristates such as those of isopropyl, butyl and cetyl, hexyl stearate, the triglycerides of C8-C18 fatty acids, cetyl ricinoleate, stearyl octanoate (PurCellin oil), cetostearyl 2-ethyl hexanoate, the esters of fatty acids and of glycerol, hydrogenated polyisobutene, and combinations thereof.

As butter, mention can be made of shea butter, cocoa butter, avocado butter, mango butter, olive butter, almond butter, and combinations thereof.

As mineral oils, mention can be made of Vaseline oil, paraffin and combinations thereof.

The butters, waxes, and vegetable and mineral oils can act as emollients by contributing lipids to the skin.

As emulsifiers, mention can be made of cetearyl alcohol, cetyl alcohol, soy lecithin, sugar esters and combinations thereof.

As vitamin, mention can be made of vitamin C, vitamin E, vitamin A and combinations thereof.

As vitamin A, mention can be made of retinol, retinoic acid or retinaldehyde.

As vitamin precursor, mention can be made of provitamin A.

As natural plant extracts, mention can be made of extracts selected from among rosemary, avocado, pumpkin, sweet almond, Swiss alpine mallow, alpine flax, castor oil plant, scotch thistle, sea fennel, Indian jasmine, mangosteen, chaste tree, apricot, oat plant, aronia berries, cornflower, wheat, chamomile, cucumber, sunflowers, yellow gentian, malt, ivy, summer lilac, lemon balm, honey, biennial evening primrose, nettle, apple, horsetail, rosehip, white fir, sage, elderberry, thyme, lemon verbena, alpine wormwood, alpine willowherb (Fleischer's willowherb), and combinations thereof.

When the composition of the invention comprises an extract of Edelweiss, the composition of the invention may also comprise at least one plant extract selected from among Swiss alpine mallow, summer lilac, alpine wormwood, alpine willowherb (Fleischer's willowherb), alpine flax, chaste tree, and combinations thereof.

As active peptides, mention can be made of palmitoyl pentapeptide-4, acetyl hexapeptide-3, mu-conotoxin CnIIIc, copper tripeptide-3, and combinations thereof.

As anti-dark spot active substances, mention can be made of resorcinol, PhE-resorcinol, gingko, ferulic acid, and combinations thereof.

As anti-UVA filters, mention can be made of terephthalyidene dicamphor sulfonic acid, butyl methoxydibenzoylmethane, and combinations thereof.

As anti-UVB filters, mention can be made of homosalate, benzophenones, phenylbenzimidazole sulfonic acid, octocrylene, ethylhexyl methoxicinnamate, ethylhexine triazone, 4-methylbenzylidene camphor, salicylates, ethylhexyl or octyl dimethyl, titanium dioxide, zinc oxide, polysilicones, and combinations thereof.

In one preferred embodiment, the composition of the invention may further comprise hyaluronic acid, glycolic acid, fruit acids, anti-UVA filters, anti-UVB filters, vitamins, vitamin precursors, natural plant extracts, active peptides, and combinations thereof.

The composition of the invention can be in the form of a gel, a solution, water-in-oil emulsion, oil-in-water emulsion, an oil, cream, balm, ointment, liposomal dispersion, or a liniment.

The composition of the invention can be intended to be applied to the skin. Preferably, the composition of the invention can be intended to be applied to the face or body, more preferably to the face.

The composition of the invention for application thereof to the face can be a serum, a universal cream, day cream, night cream, eye contour cream, a makeup removing lotion, makeup removing milk, makeup removing oil, makeup removing balm, a cosmetic mask, toning lotion, exfoliating product, cleansing gel, cleansing foam, cleansing milk, cleansing lotion.

The composition of the invention for application thereof to the body can be a body milk, body balm, body cream, a soap, shower gel, shower oil, a body scrub.

Preferably, the composition of the invention is a serum for the face and/or for the eye contour.

Advantageously, the pH of the composition of the invention can lie between 4 and 9, preferably between 5 and 7. Therefore, the composition of the invention has a pH that is compatible with the pH of the epidermis.

A further subject of the invention is a non-therapeutic cosmetic method to treat the signs of ageing, comprising the application to the surface of the skin of a composition of the invention such as defined above.

The cosmetic composition of the invention can be prepared using any technique well known to persons skilled in the art.

Method for Preparing the Derivatives

The derivatives of formula (I) or of formula (Ia) can be prepared with any method well known to persons skilled in the art.

Method for Preparing Compounds of Formula (I)

The derivatives of formula (I) can be prepared with the method described in international application WO 2017/024255A1 and patent U.S. Pat. No. 10,611,790 B2.

In particular, the derivatives of formula (I) and alpha-NMN can be prepared following the method described below.

In particular, the compounds of formula (I) disclosed herein can be prepared as described below from the substrates A-E. It is to be understood by persons skilled in the art that these reaction schemes are by no means intended to be limiting and that variations thereto may be made without departing in spirit and scope from the present invention.

According to one embodiment, the invention relates to a method for preparing compounds of formula (I) such as described above.

The method, at a first step, involves the mono-phosphorylation of a compound having the formula (A), in the presence of phosphoryl chloride and a trialkyl phosphate, so as thereby to yield the phosphorodichloridate having the formula (B),

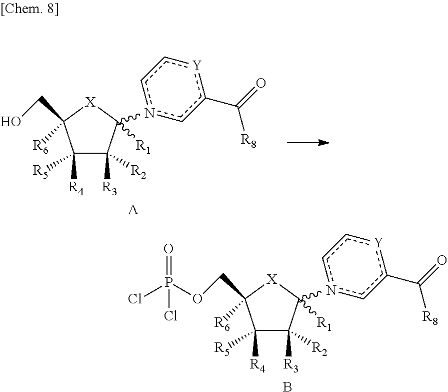

In which X, R₁, R₂, R₃, R₄, R₅, R₆, R₈, Y, [symbol] and [symbol] are such as defined above for the compounds having the formula (I).

At a second step, the phosphorodichloridate having the formula (B) is hydrolysed so as thereby to yield the phosphate having the formula (C),

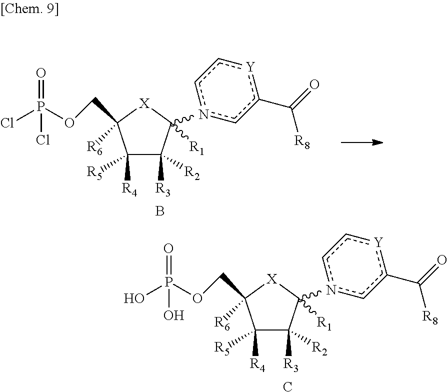

in which X, R₁, R₂, R₃, R₄, R₅, R₆, R₈, Y, [symbol] and [symbol] are as defined hereabove for the compounds having the formula (I).

According to one embodiment, the compound having the formula (A) is synthesised by means of various methods known to persons skilled in the art.

According to one embodiment, the compound having the formula (A) is synthesised by reaction of the pentose having the formula (D) with a nitrogenous derivative having the formula (E), in which R, R₂, R₃, R₄, R₅, R₆, R₇, Y, are as described hereabove for the compounds having the formula I, so as thereby to yield the compound having the formula (A),

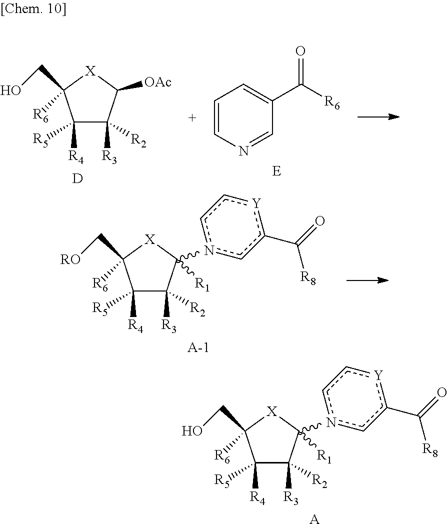

in which X, R₁, R₂, R₃, R₄, R₅, R₆, R₈, Y, [symbol] and [symbol] are such as defined above for the compounds having the formula (I).

According to one embodiment, R is a suitable protecting group known to the person skilled in the art. In one embodiment, the protecting group is selected from among triarylmethyls and/or silyls. Without limitation, some examples of triarylmethyl include trityl, monomethoxytrityl, 4,4′-dimethoxytrityl, and 4,4′,4″-trimethoxytrityl groups. Without limitation, some examples of silyl groups include trimethylsilyl, tert-butyldimethylsilyl, triisopropylsilyl, tert-butyldiphenylsilyl, tri-iso-propylsilyloxymethyl, and [2-(triméthylsilyl)ethoxy]methyl groups.

According to one embodiment, any hydroxyl group attached to the pentose is protected by an appropriate protecting group known to the person skilled in the art.

The selection and exchanging of the protecting groups are well within the scope of knowledge and expertise of the person skilled in the art. The protecting groups may also be removed by methods well known to the person skilled in the art, for example, with an acid (for example, an inorganic or organic acid), a base or a fluoride source.

In one preferred embodiment, the nitrogenous derivative having the formula (E) is coupled to the pentose having the formula (D) by a reaction in the presence of a Lewis acid so as to thereby yield the compound having the formula (A-1). Without limitation, some examples of Lewis acids include Trimethylsilyl Trifluoromethanesulfonate (TMSOTf), BF₃·OEt₂, TiCl₄ and FeCl₃.

In one embodiment, the method of the present invention additionally also comprises a reduction step of reducing the compound having the formula (A) by various methods well known to the person skilled in the art, so as thereby to yield the compound having the formula (A′) in which CH₂, and R₁, R₂, R₃, R₄, R₅, R₆, R₈, Y, [symbol] and [symbol] are such as defined hereabove for the compounds having the formula (I).

In one particular embodiment, the present invention relates to a method for preparing compounds having the formula I-A, I-C, I-E, I-G.

At a first step, the nicotinamide having the formula E is coupled to the ribose tetraacetate having the formula D by a coupling reaction in the presence of a Lewis acid, so as thereby to yield the compound having the formula A-1:

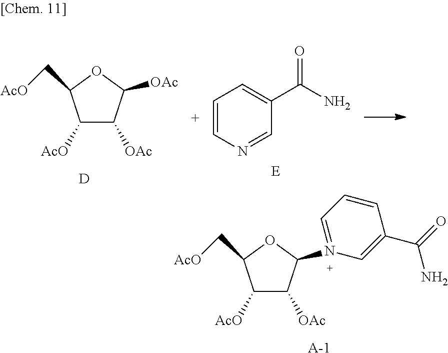

At a second step, an ammoniacal treatment of the compound having the formula A-1 is carried out, so as thereby to yield the compound having the formula I-A:

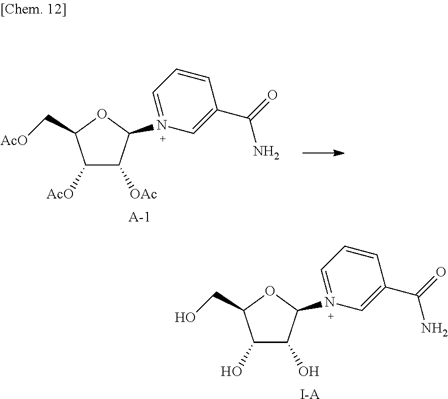

At a third step, the mono-phosphorylation of the compound having the formula I-A, in the presence of phosphoryl chloride and a trialkyl phosphate, thereby yields the phosphorodichloridate having the formula I-A′:

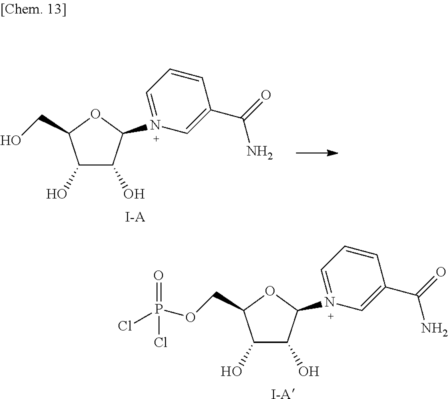

At a fourth step, the phosphorodichloridate having the formula B is hydrolysed so as thereby to yield the compound having the formula I-C:

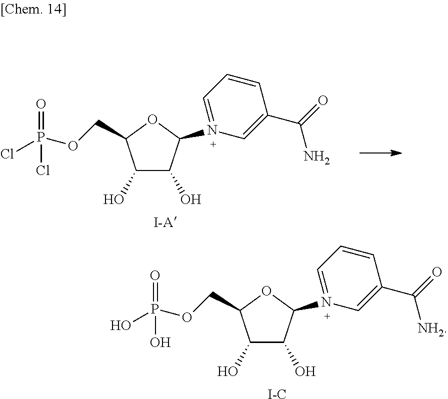

In one embodiment, a reduction step of reducing the compound having the formula I-A is carried out, so as thereby to yield the compound having the formula I-E.

The compound having the formula I-E is then mono-phosphorylated as described in the fourth step and hydrolysed so as thereby to yield the compound having the formula I-G.

According to one embodiment, the compounds having the formula (I) are selected from compounds I-A to I-B in the table below:

TABLE 1

| Compounds | |
|---|---|
| (anomers) | Structure |
| I-A (beta) | 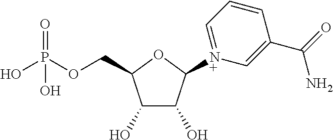 [SMILES: NC(=O)c1ccc[n+]([C@@H]2O[C@H](COP(=O)(O)O)[C@@H](O)[C@H]2O)c1] |
| I-B (alpha) | 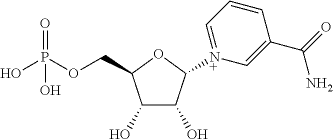 [SMILES: NC(=O)c1ccc[n+]([C@H]2O[C@H](COP(=O)(O)O)[C@@H](O)[C@H]2O)c1] |
| I-C (beta) | 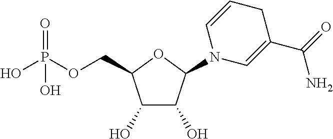 [SMILES: NC(=O)C1=CN([C@@H]2O[C@H](COP(=O)(O)O)[C@@H](O)[C@H]2O)C=CC1] |
| I-D (alpha) | 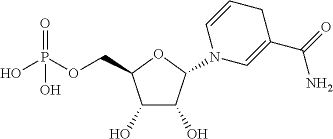 [SMILES: NC(=O)C1=CN([C@H]2O[C@H](COP(=O)(O)O)[C@@H](O)[C@H]2O)C=CC1] |
| I-E (beta) | 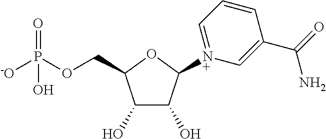 |
| I-F (alpha) | 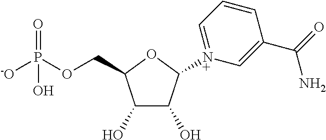 |
| I-G (beta) | 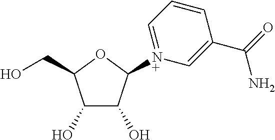 [SMILES: NC(=O)c1ccc[n+]([C@@H]2O[C@H](CO)[C@@H](O)[C@H]2O)c1] |
| I-H (alpha) | 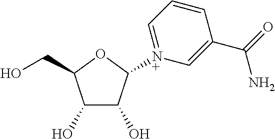 [SMILES: NC(=O)c1ccc[n+]([C@H]2O[C@H](CO)[C@@H](O)[C@H]2O)c1] |
| I-I (beta) | 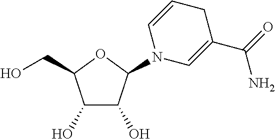 [SMILES: NC(=O)C1=CN([C@@H]2O[C@H](CO)[C@@H](O)[C@H]2O)C=CC1] |
| I-J (alpha) | 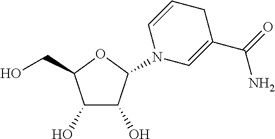 [SMILES: NC(=O)C1=CN([C@H]2O[C@H](CO)[C@@H](O)[C@H]2O)C=CC1] |

In one preferred embodiment, the compounds of the invention are the compounds of formula I-A, I-C, I-E et I-G in the Table above, or a pharmaceutically acceptable salt and/or solvate thereof. In one further preferred embodiment, the compound is the compound of formula I-C or I-D or a pharmaceutically acceptable salt and/or solvate thereof.

Synthesis of the formula I compounds in which R7 is

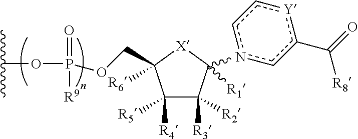

and n=1:

At a first step, the synthesis may comprise the phosphorylation of alpha-nicotinamide riboside, in the presence of phosphorus chloride and a trialkyl phosphate, to obtain a phosphorodichloridate:

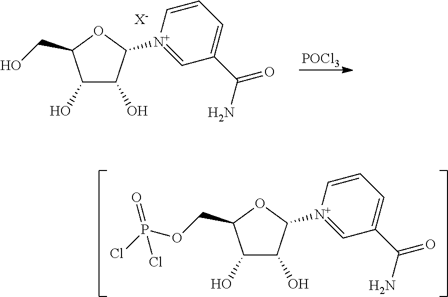

The compound I-B (alpha NMN) is then added:

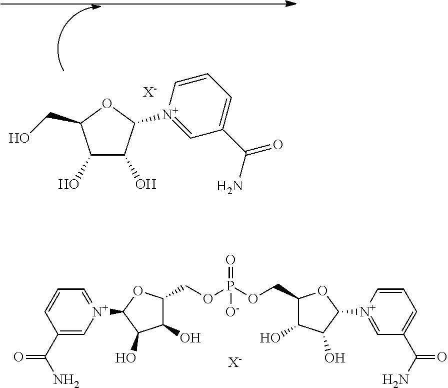

Alternatively, the compound of the invention can be prepared via activation of compound I-B through the addition of carbonyldiimidazole (CDI):

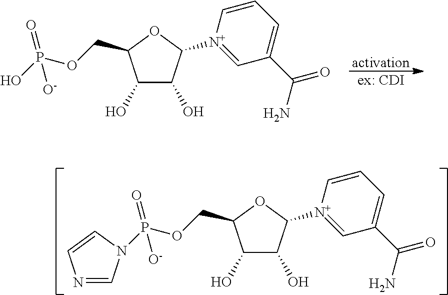

To which alpha-nicotinamide riboside is added as follows:

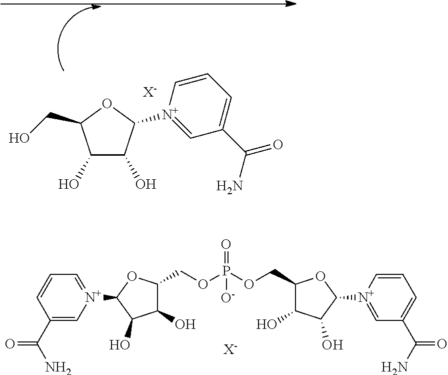

Synthesis of the formula I compounds in which R7 is

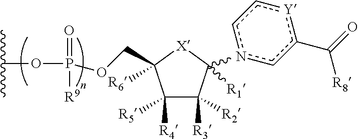

and n=3:

A compound of formula I comprising three phosphate groups can be prepared as follows. Compound I-B can be prepared through the addition of carbonyldiimidazole (CDI):

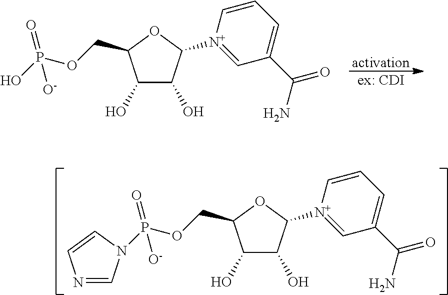

To which tertbutylamine-phosphate is added:

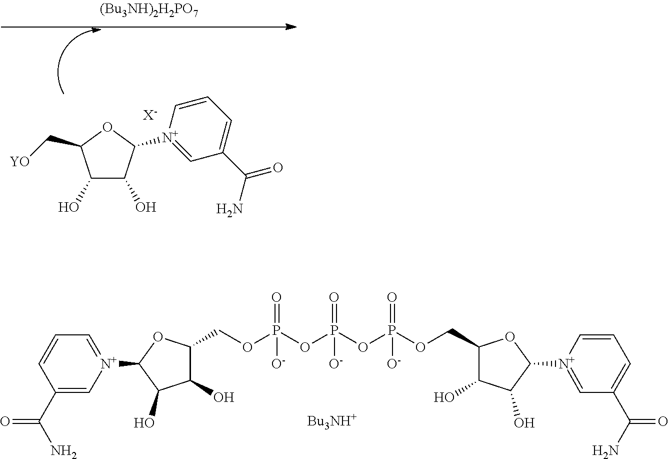

Method for Preparing the Derivatives Having the Formula (Ia)

In particular, the compounds having the formula Ia presented herein may be prepared as described below from the substrates X-XIII. It is to be understood by the average person skilled in the art that these schemes are by no means intended to be limiting and that variations thereto in terms of the detail may be made without departing in spirit and scope from the present invention.

According to one embodiment, the invention relates to a compound preparation method for preparing the compound having the formula I described hereabove.

The method consists first of all in mono-phosphorylating a compound having the formula X, in the presence of phosphoryl chloride in a trialkyl phosphate, in order to obtain the compound phophorodichloridate XI,

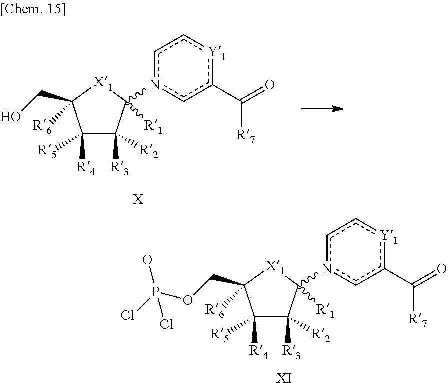

in which X′₁, R′₁, R′₂, R′₃, R′₄, R′₅, R′₆, R′₇, Y′₁, [symbol] and [symbol] are such as defined above.

At a second step, the hydrolysis of the phosphorodichloridate XI obtained at the first step gives the phosphate compound having the formula XII,

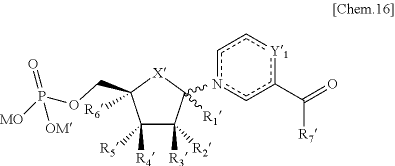

in which X′₁, R′₁, R′₂, R′₃, R′₄, R′₅, R′₆, R′₇, Y′₁, M′, [symbol] and [symbol] are such as defined above.

The phosphate compound having the formula XII obtained at the second step is then reacted with a phosphorodichloridate compound having the formula XIII obtained as described at the first step.

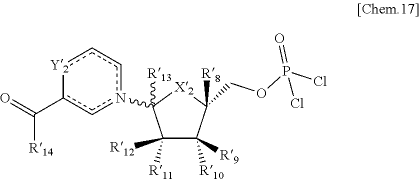

in which X′₂, R′₈, R′₉, R′₁₀, R′₁₁, R′₁₂, R′₁₃, R′₁₄, Y′₂, [symbol] and [symbol] are such as defined herein for formula Ia, in order to give the compound having the formula Ia such as described herein.

According to one embodiment, the method further comprises a reduction step of reducing the compound having the formula Ia, using various methods known to specialists, in order to give the compound having the formula Ia, where Y′₁ and Y′₂ are identical and each represent CH₂, and where X′₁, X′₂, R′₁, R′₂, R′₃, R′₄, R′₅, R′₆, R′₇, R′₈, R′₉, R′₁₀, R′₁₁, R′₁₂, R′₁₃, R′₁₄, Y′₁, Y′₂, [symbol] and [symbol] are as described herein for formula Ia.

In one variant, R is a suitable protecting group known to the person skilled in the art. Triarylmethyl and/or silyl groups are examples of suitable protecting groups. Without limitation, some examples of triarylmethyl include trityl, monomethoxytrityl, 4,4′-dimethoxytrityl, and 4,4′,4″-trimethoxytrityl. Without limitation, some examples of silyl groups include trimethylsilyl, tert-butyldimethylsilyl, triisopropylsilyl, tert-butyldiphenylsilyl, tri-iso-propylsilyloxymethyl, and [2-(triméthylsilyl)éthoxy]methyl.

According to one representation, any hydroxy group attached to the pentose ring is protected by a suitable protecting group known to the person skilled in the art.

The selection and exchanging of the protecting groups are well within the scope of knowledge and expertise of the person skilled in the art. Any protecting group may also be removed by methods known in the art, for example, with an acid (for example, an inorganic or organic acid), a base or a fluoride source.

According to one preferred embodiment, the nitrogen compounds having the formula XV are added to the pentose XIV by a coupling reaction in the presence of a Lewis acid in order to give the compound having the formula X-1. Without limitation, some examples of suitable Lewis acids include Trimethylsilyl Trifluoromethanesulfonate (TMSOTf), BF₃·OEt₂, TiCl₄ and FeCl3.

According to one specific embodiment, the invention relates to a compound preparation method for preparing the compound having the formula VIII,

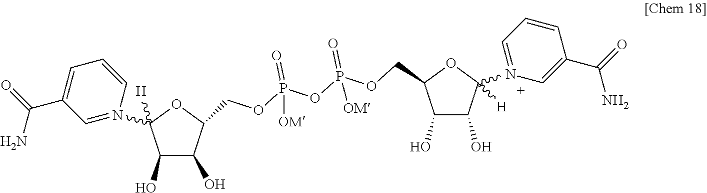

or the pharmaceutically acceptable salts and/or solvates thereof.

At a first step, the nicotinamide having the formula XV is added to the ribose tetraacetate XIV, by a coupling reaction in the presence of a Lewis acid, in order to give the compound having the formula X-1:

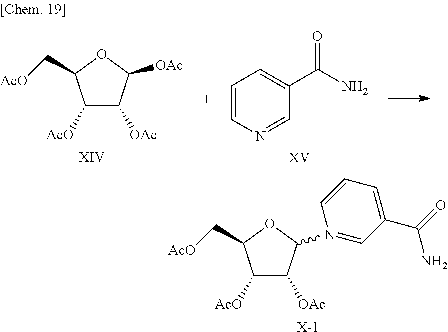

At a second step, an ammoniacal treatment of the compound having the formula X-1 gives the compound having the formula X:

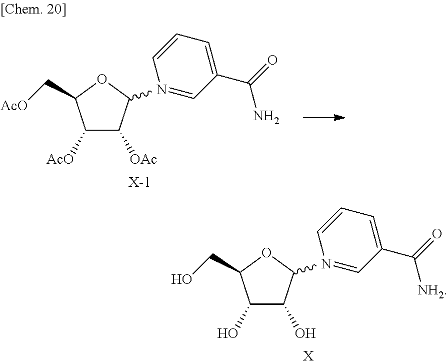

At a third step, the mono-phosphorylation of a compound having the formula X, in the presence of phosphoryl chloride in a trialkyl phosphate, gives the compound phosphorodichloridate XI:

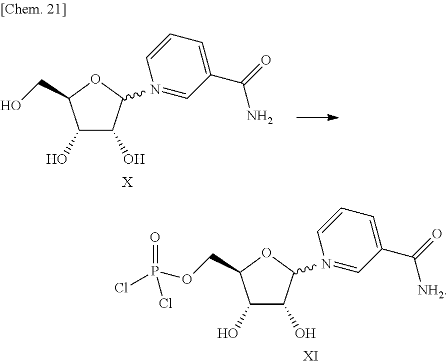

At a fourth step, the phosphorodichloridate compound XI obtained at the third step is partially hydrolysed in order to give the phosphate compound having the formula XII:

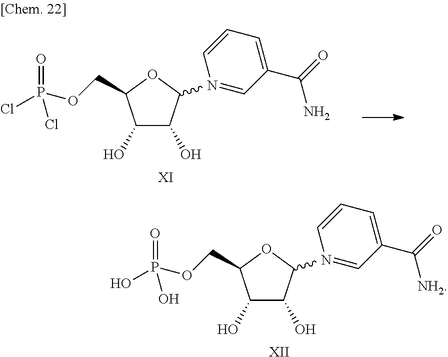

At a fifth step, the phosphate compound having the formula XII obtained at the fourth step is then reacted with the phosphorodichloridate compound having the formula XI obtained as described at the third step, in order to obtain the compound having the formula VIII.

According to another specific implementation embodiment, the invention relates to a compound preparation method for preparing the compound having the formula IX,

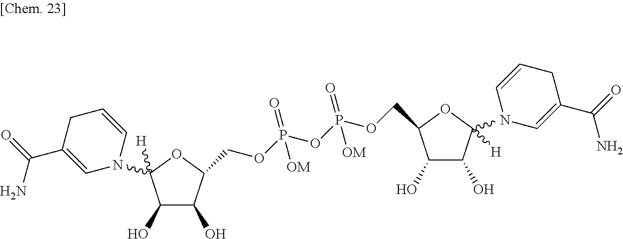

or the pharmaceutically acceptable salts and/or solvates thereof.

According to one variant, the compound having the formula IX is obtained from the compound having the formula VIII, which is synthesised beforehand as described hereabove.

In this embodiment, the compound having the formula IX is obtained by reducing the compound having the formula VIII, using a suitable reducing agent known to the specialised person skilled in the art, in order to give the compound having the formula IX.

According to one embodiment, the preferred compounds of the invention are the compounds Ia-A to Ia-I in Table 2:

TABLE 2

| Compound | Structure |
|---|---|
| Ia-A (beta, beta) | 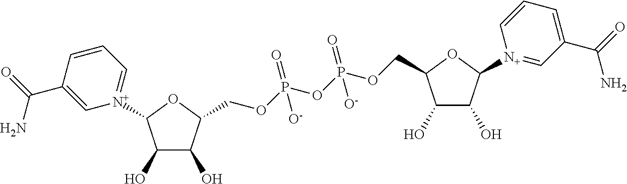 [SMILES: NC(=O)c1ccc[n+]([C@@H]2O[C@H](COP(=O)([O-])OP(=O)([O-])OC[C@H]3O[C@@H]([n+]4cccc(C(N)=O)c4)[C@H](O)[C@@H]3O)[C@@H](O)[C@H]2O)c1] |
| Ia-B (beta, alpha) | 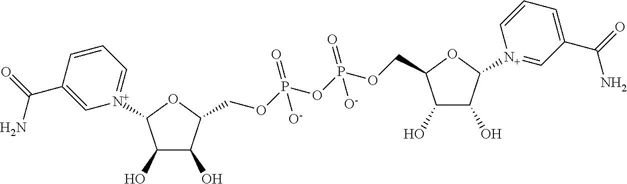 [SMILES: NC(=O)c1ccc[n+]([C@@H]2O[C@H](COP(=O)([O-])OP(=O)([O-])OC[C@H]3O[C@H]([n+]4cccc(C(N)=O)c4)[C@H](O)[C@@H]3O)[C@@H](O)[C@H]2O)c1] |
| Ia-C (alpha, alpha) | 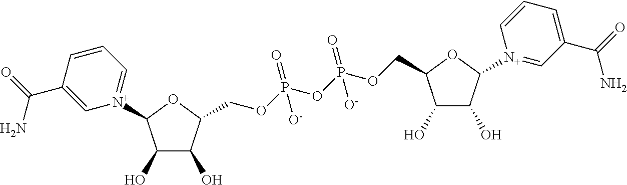 [SMILES: NC(=O)c1ccc[n+]([C@H]2O[C@H](COP(=O)([O-])OP(=O)([O-])OC[C@H]3O[C@H]([n+]4cccc(C(N)=O)c4)[C@H](O)[C@@H]3O)[C@@H](O)[C@H]2O)c1] |
| Ia-D (beta, beta) | 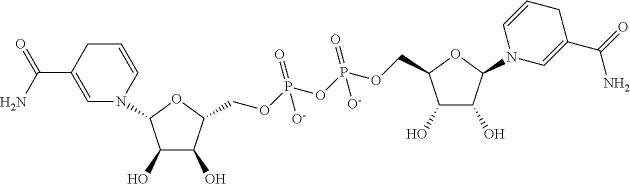 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 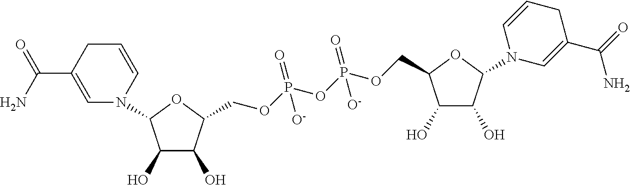 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 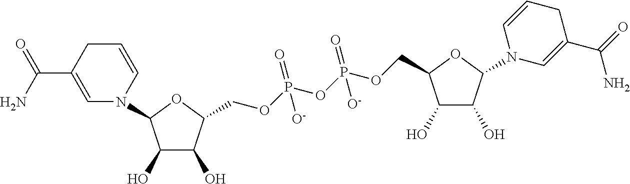 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 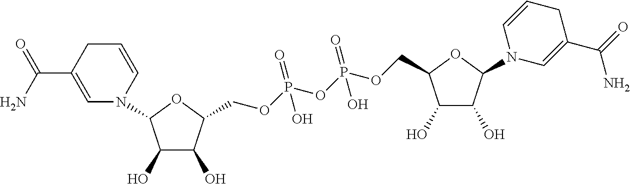 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 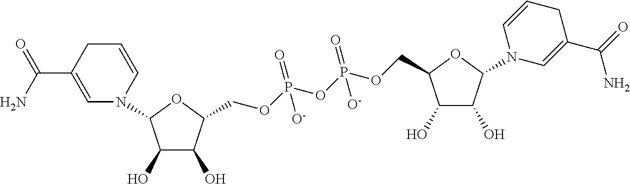 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 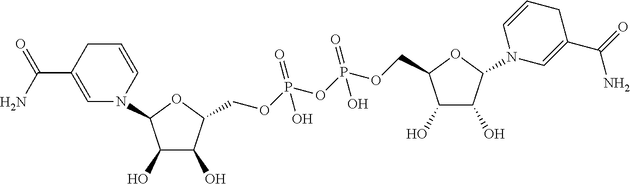 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

### FIGURES

FIG. 1 is a graph showing the change in parameters Ra, Rz and Rt as a function of dry, normal or oily skin type and as a function of treatment with the composition comprising NMN of the invention or with the same cream without NMN as negative control.

FIG. 2 is a graph showing improvement in thickness of the skin as a function of dry, normal or oily skin type and as a function of treatment with the composition comprising NMN of the invention or with the same cream without NMN as negative control.

FIG. 3 is a graph showing the improvement in firmness and elasticity of the skin as a function of dry, normal or oily skin type and as a function of treatment with the composition comprising NMN of the invention or with the same cream without NMN as negative control.

FIG. 4 is a graph showing changes in wrinkles, fine lines, swelling of the eyelid, dark under-eye circles, and the tired appearance of the skin for all skin types as a function of treatment with the composition comprising NMN of the invention or with the same cream without NMN as negative control.

FIG. 5 is a schematic illustrating the principle of fringe projection.

FIG. 6 is a schematic illustrating the principle of calculating parameter Ra.

FIG. 7 is a schematic illustrating the principle of calculating parameters Rz and Rt.

FIG. 8 gives photographs of the Bazin grading scale for assessing crow's feet wrinkles and under-eye wrinkles and fine lines.

FIG. 9 shows a skin deformation curve obtained with the Cutometer®.

FIG. 10 gives photographs of the Bazin grading scale for assessing swelling of the lower eyelid.

FIG. 11 gives photographs of the Bazin grading scale for assessing under-eye dark circles.

FIG. 12 is a schematic illustrating the principle of analysis with the Glossymeter.

FIG. 13 is a graph giving the results on skin hydration.

FIG. 14 is a graph giving the results on skin elasticity.

FIG. 15 is a graph giving the results on skin roughness.

FIG. 16 is a graph giving the results on the volume of skin wrinkles.

FIG. 17 is a graph showing the change in hydration, with the face serum of the invention.

FIG. 18 is a graph showing the change in elasticity, with the face serum of the invention.

FIG. 19 is a graph showing the change in roughness, with the face serum of the invention.

FIG. 20 is a graph showing the change in wrinkle volume, with the face serum of the invention.

FIG. 21 is a graph showing the change in hydration, with the eye contour serum of the invention.

FIG. 22 is a graph showing the change in elasticity, with the eye contour serum of the invention.

FIG. 23 is a graph showing the change in roughness, with the eye contour serum of the invention.

FIG. 24 is a graph showing the change in wrinkle volume, with the eye contour serum of the invention.

FIG. 25 is a graph showing the change in the volume of under-eye bags, with the eye contour serum of the invention.

FIG. 26 is a graph showing the change in eye contour erythema, with the eye contour serum of the invention.

FIG. 27 is a graph showing the change in melanin content of the eye contour, with the eye contour serum of the invention.

### EXAMPLES

In the remainder of the present description, the examples are provided to illustrate the invention and are not at all intended to limit the scope thereof.

In the examples below, the weight percentage of an ingredient is calculated as follows: weight percentage of ingredient (%)=(ingredient weight)×100/(total weight of composition)

### Example 1

An example of the cosmetic composition of the invention was tested on two groups of healthy volunteers composed of women aged between 45 and 65 years, having dry skin (denoted S), normal skin (denoted N) or mixed type (denoted M). The phototype of the participants was between phototype II and IV. The first group, twice daily, applied to the face a cosmetic composition comprising 2% NMN. The second group, twice daily, applied to the face the same composition not comprising NMN as negative control. The study lasted 28 days. The composition of the invention was prepared using the control composition and mixing therewith the NMN in zwitterion form up to a final weight concentration of 2%. The study lasted 28 days.

TABLE 3

| | | Function of | Weight |
|---|---|---|---|
| INCI EU | CAS | Ingredient | % |
| Aqua | 7732-18- | solvent | 70 |
| | 5 | | |
| Caprylic/Capric | 73398- | moisturizing/ | 2.00 |
| Triglyceride | 61-5 | masking/fragrancing/ | |
| | | humectant/solvent | |
| Nicotinamide | 1094-61- | emollient | 2.00 |
| mononucleotide | 7 | | |
| Glycerin | 56-81-5 | denaturing/ | 5.00 |
| | | humectant/ | |
| | | fragrancing/solvent | |
| Butylene Glycol | 107-88-0 | humectant/masking/ | 3.00 |
| | | solvent/viscosity | |
| | | controlling | |
| Glyceryl Stearate SE | 11099- | emulsifying | 1.00 |
| | 07-3 | | |
| Propylene Glycol | 41395- | moisturizing/viscosity | 3.00 |
| Dipelargonate | 83-9 | controlling | |
| Pentylene Glycol | 5343-92- | humectant/solvent | 2.00 |
| | 0 | | |
| Dicaprylyl Carbonate | 1680-31- | moisturizing/ | 2.00 |
| | 5 | humectant | |
| Butyrospermum | 194043- | humectant/viscosity | 2.00 |
| Parkii Butter (Shea | 92-0 | controlling/emollient | |
| Butter) | | | |
| Hydrogenated Palm | 93334- | moisturizing/ | 2.00 |
| Kernel Glycerides | 20-4 | humectant/viscosity | |
| | | controlling | |
| Glyceryl Stearate | 31566- | moisturizing/ | 1.00 |
| | 31-1 | emulsifying | |
| Sodium Citrate | 6132-04- | buffering | 0.30 |
| | 3 | | |
| Hydroxyethyl | 111286- | emulsion stabilising/ | 0.40 |
| Acrylate/Sodium | 86-3 | viscosity controlling | |
| Acryloyldimethyl | | | |
| Taurate Copolymer | | | |
| Polyacrylate | / | emulsion stabilising/ | 0.50 |
| Crosspolymer-6 | | viscosity controlling | |
| Steareth-21 | 9005-00- | cleansing/ | 0.50 |
| | 9 | emulsifying/ | |
| | (Generic) | surfactant | |
| Glyceryl Caprylate | 26402- | moisturizing/ | 0.30 |
| | 26-6 | emulsifying | |
| Xanthan Gum | 11138- | binder/emulsifying/ | 0.20 |
| | 66-2 | emulsion stabilising/ | |
| | | gelling/humectant/ | |
| | | surfactant/viscosity | |
| | | controlling | |
| Chlorphenesin | 104-29-0 | preserving | <0.1 |
| Tocopherol | / | antioxidant/masking/ | 0.2 |
| | 10191- | humectant | |
| | 41-0 | | |
| | 59-02-9 | | |
| Hydrogenated Palm | 91744- | moisturizing/ | 2.00 |
| Glycerides | 66-0 | emulsifying/ | |
| | | humectant/viscosity | |
| | | controlling | |
| Ethylhexylglycerin | 70445- | humectant | 0.2 |
| | 33-9 | | |
| Citric Acid | 77-92-9 | buffering/masking | <0.1 |
| Glycine Soja Oil | 8001-22- | moisturizing/ | <0.1 |
| | 7 | fragrancing/skin care | |
| Polysorbate 60 | 9005-67- | emulsifying/ | <0.1 |
| | 8 | surfactant | |
| Sorbitan Isostearate | 71902- | emulsifying | <0.1 |
| | 01-7 | | |

Different signs of ageing were assessed in the participants and in particular skin roughness via fringe projection, skin firmness and skin elasticity via Cutometry®, skin density via DermaScan® ultrasound, swelling of the lower eyelid, dark under-eye circles and tired appearance being clinically assessed by an expert. These parameters were measured in the patients at Day 0, at the time of inclusion in the study and before any treatment, and at Day 28 at the end of the study. The different parameters measured at Day 28 were compared with those measured at Day 0 for each group and the parameters measured at Day 28 were compared between the group treated with NMN and the control group. The results were evaluated with a Student's test on EXCEL software (Microsoft) and SAS 9.4.

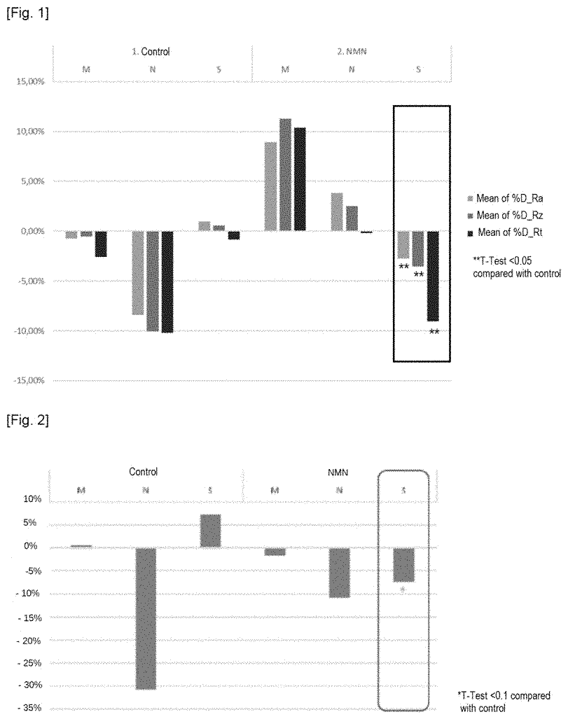

In FIG. 1, the composition of the invention allowed a significant reduction in parameters Ra, Rz and Rt in particular for the dry skin type. The composition of the invention therefore allows skin roughness to be reduced and the skin to be smoothed, in particular for dry skin types.

In FIG. 2, the composition of the invention allowed an increase in skin thickness of about 5% for all skin types taken together. However, the composition gives a statistically significant increase in the dry skin types. One of the signs of ageing is a decrease in skin thickness. Therefore, the composition of the invention allows the prevention or treatment of a sign of ageing and in particular allows skin thickness to be increased.

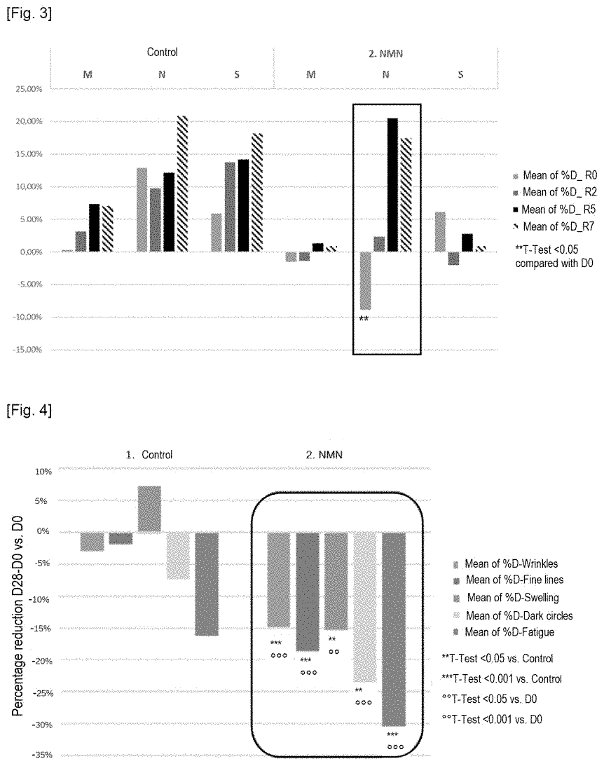

In FIG. 3, the composition of the invention allowed a significant increase in the firmness of normal skin.

FIG. 4 shows the changes in different signs of ageing, irrespective of skin type. An improvement in wrinkles, fine lines, swelling of the lower eyelid, and dark circles was determined by the reduction of each of these parameter values. Reduction (as %) is denoted % D and is calculated as follows: % D=(value obtained on the area treated with the product at Day 28 −value obtained on the area treated with the product at D0)×100/(value obtained on the area treated with the product at D0). In FIG. 4, the composition of the invention allows a significant decrease in the size and depth of wrinkles and fine lines, and in swelling of the lower eyelid, in the size of under-eye dark circles and the tired appearance of the skin.

After use over 28 days, a significant improvement of 11% in the skin radiance of the participants was measured with the Glossymeter after use of the cosmetic composition of the invention comprising NMN (p=0.032). The composition of the invention comprising NMN therefore leaves a more radiant skin which therefore appears less tired and contributes towards the anti-age effect.

The participants were also invited to reply to a self-assessment questionnaire relating to the signs of ageing. As can be seen below, the majority of participants were satisfied with the composition of the invention:

TABLE 4

| | Percentage of participants in |
|---|---|
| | agreement with the assertion (%) |
| The product is moisturizing | 91 |
| My skin is firmer | 83 |
| My skin appears denser | 83 |
| My skin appears plumper | 69 |
| My facial features are less tired- | 82 |
| looking | |
| My skin is softer | 96 |
| My skin is smoother | 86 |
| The skin feels comfortable and | 78 |
| restored | |
| The product leaves a glow on the skin | 91 |
| The product restores radiance to the | 87 |
| skin | |
| The wrinkles and fine lines of crow’s | 52 |
| feet have faded | |
| The sigs of ageing have decreased | 65 |

The composition in Example 1 was also tested on 24 participants of Afro-American type aged 40-65 years, all having wrinkles, fine lines and crow's feet at the outer corner of the eyes. One half of these women had dry skin and the other half had normal skin. Measurements of wrinkles, dark circles, swelling of the lower eyelid, tired appearance of the skin (namely loss of skin radiance) and skin sagging (comprising loss of firmness and loss of elasticity) as indicated above were performed at days D0, D28 and D56. The results were obtained by comparing the measurements obtained at D28 with those at D0, and comparing D56 results with those at D0. The results were analysed using a Wilcoxon test and were considered significant for p<0.05.

As shown in Table 5 below, the cream of the invention allows an improvement in all the signs of ageing, whether for dry skin or normal skin in Afro-American skin types:

TABLE 5

| | Mean ± | | | | % subjects |
|---|---|---|---|---|---|
| | standard | | % | | showing |
| | deviation | | reduction | p | improvement |
| Wrinkles | D28-D0 | −0.3 ± 0.1 | −10% | 0.0078 | 33% |
| | D56-D0 | −0.5 ± 0.1 | −14% | 0.0039 | 38% |
| Swelling | D28-D0 | −0.3 ± 0.1 | −18% | 0.0156 | 29% |
| eyelid | | | | | |
| of lower | D56-D0 | −0.5 ± 0.1 | −29% | 0.0039 | 38% |
| Dark | D28-D0 | −0.3 ± 0.1 | −18% | 0.0078 | 33% |
| circles | D56-D0 | −0.5 ± 0.1 | −26% | 0.0020 | 42% |
| Skin | D28-D0 | −0.7 ± 0.2 | −17% | 0.0002 | 54% |
| sagging | D56-D0 | −0.9 ± 0.2 | −22% | <0.0001 | 67% |
| Fatigue | D28-D0 | −0.4 ± 0.1 | −15% | <0.0001 | 71% |
| | D56-D0 | −0.6 ± 0.1 | −21% | <0.0001 | 75% |

The composition of Example 1 was also tested on 21 participants of Afro-American type aged 40-65 years, all having wrinkles, fine lines and crow's feet wrinkles at the outer corner of the eyes. One half of these women had dry skin and the other half had normal skin. Measurements on wrinkles, dark circles, swelling of the lower eyelid, tired appearance of the skin (namely loss of skin radiance) and skin sagging (comprising loss of firmness and loss of elasticity), as indicated above, were taken on days D0, D28 and D56. The results were obtained by comparing the measurements obtained at D28 with those at D0, and comparing D56 results with those at D0. The results were analysed using a Wilcoxon test and considered to be significant for p<0.05.

As shown in Table 6 below, the cream of the invention allows an improvement in all the signs of ageing whether for dry skin or normal skin, for Afro-American skin types:

TABLE 6

| | Mean ± | | | | % subjects |
|---|---|---|---|---|---|
| | standard | | % | | showing |
| | deviation | | reduction | p | improvement |
| Wrinkles | D28-D0 | −0.3 ± 0.1 | −24% | 0.0002 | 62% |
| | D56-D0 | −0.4 ± 0.1 | −33% | 0.0002 | 68% |
| Swelling | D28-D0 | −0.2 ± 0.1 | −16% | 0.0078 | 38% |
| eyelid | | | | | |
| of lower | D56-D0 | −0.4 ± 0.1 | −44% | 0.0003 | 74% |
| Dark | D28-D0 | −0.3 ± 0.1 | −17% | 0.0000 | 57% |
| circles | D56-D0 | −0.6 ± 0.1 | −37% | <0.0001 | 84% |
| Skin | D28-D0 | −0.2 ± 0.1 | −5% | 0.1250 | 29% |
| sagging | D56-D0 | −0.4 ± 0.1 | −10% | 0.0352 | 42% |
| Fatigue | D28-D0 | −0.3 ± 0.1 | −11% | <0.0001 | 95% |
| | D56-D0 | −0.9 ± 0.1 | −41% | <0.0001 | 100% |

The composition of the invention therefore allows a significant reduction to be obtained in the signs of ageing.

The use of NMN and of compositions comprising the same according to the invention therefore allows the signs of ageing to be reduced, in particular in dry and normal skin types.

### Example 2—Face Cream with Hyaluronic Acid

An example of a cosmetic composition of the invention was tested on a group of 20 healthy volunteers composed of women aged between 45 and 60 years. Each volunteer was self-assessed before and after: the comparison was made between D0 and D7, D28 or D56.

The volunteers, twice daily, applied to the face a cosmetic composition comprising 2% NMN and 0.35% hyaluronic acid in the form of sodium hyaluronate as shown in Table 7 below:

TABLE 7
Face cream

| Ingredients (INCI | CAS | | Weight |
|---|---|---|---|
| name) | number | Role | percent |
| Aqua | 7732-18-5 | Solvent | q.s. to 100% |
| Glycerine | 56-81-5 | Humectant, | 4.5 |
| | | solvent | |
| Squalane | 111-01-3 | Emollient | 4.0 |
| Glyceryl Stearate | 11099-07-3 | Emulsifying | 3.0 |
| SE | | | |
| Propanediol | 504-63-2 | Viscosity | 3.0 |
| | | controlling, solvent | |
| Propylene Glycol | 41395-83-9 | Emollient, | 3.0 |
| Dipelargonate | | viscosity | |
| | | controlling | |
| Caprylic/Capric | 73398-61-5 | Emollient, solvent, | 2.0 |
| Triglyceride | | fragrancing | |
| Nicotinamide | 1094-61-7 | Active cosmetic | 2.0 |
| Mononucleotide | | substance | |
| Pentylene Glycol | 5343-92-0 | Solvent, Emollient | 2.0 |
| Dicaprylyl | 1680-31-5 | Emollient | 1.99 |
| Carbonate | | | |
| C10-18 | 85665-33-4 | Emollient, solvent | 1.5 |
| Triglycerides | | | |
| Butyrospermum | 194043-92- | Emollient, | 1.5 |
| Parkii Butter | 0 | viscosity | |
| | | controlling | |
| Glyceryl Stearate | 31566-31-1 | Emollient, | 0.75 |
| | | emulsifying | |
| Polyacrylate | / | Emulsion | 0.5 |
| Crosspolymer-6 | | stabilising, | |
| | | viscosity | |
| | | controlling | |
| Silica | 7631-86-9 | Absorbent, | 0.5 |
| | | viscosity | |
| | | controlling | |
| Steareth-21 | 9005-00-9 | Surfactant, | 0.5 |
| | (Generic) | emulsifying | |
| Hydroxyethyl | 111286-86- | Emulsion | 0.4 |
| Acrylate/Sodium | 3 | stabilising, | |
| Acryloyldimethyl | | viscosity | |
| Taurate | | controlling | |
| Copolymer | | | |
| Isomalt | 64519-82-0 | Humectant | 0.372 |
| Sodium | 9067-32-7 | Humectant, | 0.35 |
| Hyaluronate | | emollient | |
| Glyceryl | 26402-26-6 | Emollient, | 0.30 |
| Caprylate | | emulsifying | |
| Sodium Citrate | 6132-04-3 | Buffering agent | 0.30 |
| | | chelating | |
| Xanthan Gum | 6132-04-3 | Binder, | 0.20 |
| | | emulsifying, | |
| | | surfactant, | |
| | | viscosity | |
| | | controlling, | |
| | | emollient | |
| Ethylhexylglycerin | 70445-33-9 | Lipid-replenishing | 0.149850 |
| Tocopherol | 10191-41-0 | Antioxidant, | 0.140900 |
| | 59-02-9 | emollient | |
| Sodium | 1310-73-2 | Buffering agent | 0.072 |
| Hydroxide | | | |
| Glycine Soja Oil | 8001-22-7 | Emollient, | 0.060 |
| | | fragrance | |
| Citric Acid | 77-92-9 | Antioxidant | 0.055515 |
| Polysorbate 60 | 9005-67-8 | Chelating/buffering | 0.030 |
| | | agent | |
| Sorbitan | 71902-01-7 | Emulsifying | 0.030 |
| Isostearate | | | |
| Sodium Benzoate | 532-32-1 | Preserving | 0.0060 |
| Lecithin | 8002-43-5 | Emulsifying | 0.00320 |
| Rhododendron | 90106-21-1 | Emollient | 0.00320 |
| Ferrugineum Leaf | | | |
| Cell Culture | | | |
| Extract | | | |

The study lasted 56 days. Follow-up visits were organised at D0, D7, D28 and D56. The improvement in the parameters of skin ageing signs were measured at the time of these four visits.

Different signs of ageing were assessed in the participants and in particular skin roughness via fringe projection (see FIG. 1), skin firmness and skin elasticity via Cutometry®. These parameters were measured in the patients at Day 0 at the time of study inclusion and before any treatment, at Day 7, at Day 28 in the middle of the study, and at Day 56 at the end of the study. The different parameters were also compared between the different measurement dates to evaluate the continuity of improvement. Wrinkle volume and assessment of skin roughness were evaluated using Antera 3D® software. The results were analysed with a Student's test on EXCEL software (Microsoft) and SAS 9.4.

With regard to moisturizing, the measured values were as follows (see FIG. 13):

TABLE 8

| Day of measurement | Mean | Standard deviation |
|---|---|---|
| T0 | 53.3 | 5.0 |
| T7 | 58.3 | 5.5 |
| T28 | 59.2 | 3.9 |
| T56 | 59.8 | 4.0 |

Compared with the base value (TO), moisturization of the skin is improved by (see FIG. 13)
9%, after 7 days of use of the product (p<0.05);
11%, after 28 days of use of the product (p<0.05); and
12%, after 56 days of use of the product (p<0.05).

There was also recorded a statistically significant increase in skin moisturization of:
3% between the values at T7 (after 7 days' treatment) and T56 (after 56 day's treatment) (p<0.05); and
1% between the values at T28 (after 28 days' treatment) and T56 (after 56 days' treatment) (p<0.05).

The statistical significance of the values is given below:

TABLE 9
P value (statistically significant for p < 0.05)

| | T0 | T7 | T28 |
|---|---|---|---|
| T7 | 2.083E−07 | / | / |
| T28 | 2.274E−09 | 0.1518291 | / |
| T56 | 7.913E−11 | 0.0321557 | 0.0010513 |

The composition of the invention therefore allows moisturization of the skin to be significantly improved on and after the first week.

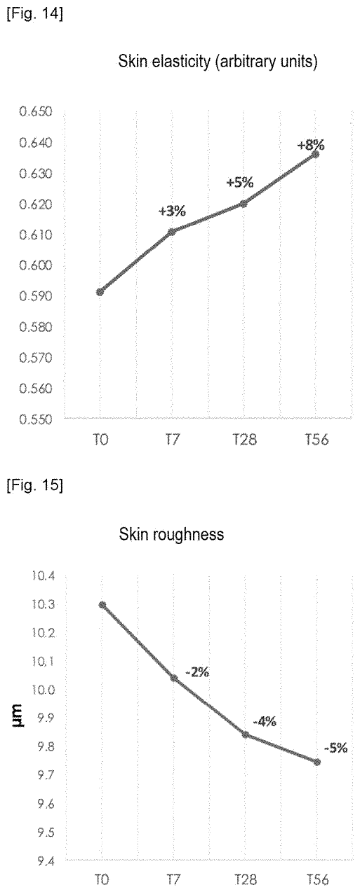

Regarding elasticity, the measured values are the following (see FIG. 14):

TABLE 10

| Day of measurement | Mean | Standard deviation |
|---|---|---|
| T0 | 0.591 | 0.056 |
| T7 | 0.611 | 0.055 |
| T28 | 0.620 | 0.044 |
| T56 | 0.636 | 0.046 |

Compared with the base value (T0), skin elasticity was improved by:
3%, after 7 days of use of the product (p<0.05);
5%, after 28 days of use of the product (p<0.05); and
8%, after 56 days of use of the product (p<0.05).

There was also recorded a statistically significant increase in skin elasticity of:
4% between the values at T7 (after 7 days' treatment) and T56 (after 56 days' treatment) (p<0.05); and
3% between the values at T28 (after 28 days' treatment) and T56 (after 56 days' treatment) (p<0.05).

The statistical significance of the values is given below:

TABLE 11
p value (statistically significant for p < 0.05)

| | T0 | T7 | T28 |
|---|---|---|---|
| T7 | 5.691E−05 | / | / |
| T28 | 0.00044102 | 0.08746976 | / |
| T56 | 1.5745E−06 | 0.00011698 | 0.00013191 |

The composition of the invention therefore allows a significant improvement in elasticity of the epidermis to be obtained on and after the first week.

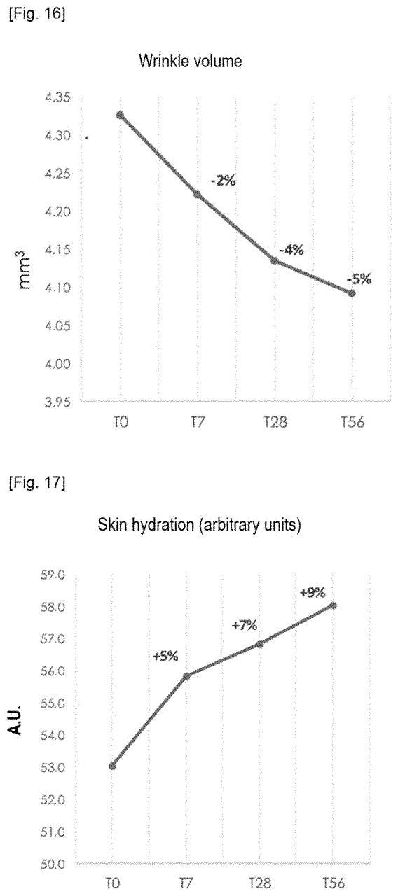

Regarding skin roughness, the measured values are the following (see FIG. 15):

TABLE 12

| Day of measurement | Mean | Standard deviation |
|---|---|---|
| T0 | 10.3 | 2.2 |
| T7 | 10.0 | 2.1 |
| T28 | 9.8 | 2.0 |
| T56 | 9.7 | 2.0 |

Compared with the base value (TO), skin roughness was improved by:
2%, after 7 days of use of the product (p<0.05);
4%, after 28 days of use of the product (p<0.05); and
5%, after 56 days of use of the product (p<0.05).

There was also recorded a statistically significant decrease in skin roughness of:
2% between the values at T7 (after 7 days' treatment) and T28 (after 38 days' treatment) (p<0.05);
3% between the values at T7 (after 7 days' treatment) and T56 (after 56 days' treatment) (p<0.05); and
1% between the values at T28 (after 28 days' treatment) and T56 (after 56 days' treatment) (p<0.05).

The statistical significance of the values is given below:

TABLE 13
p value (statistically significant for p < 0.05)

| | T0 | T7 | T28 |
|---|---|---|---|
| T7 | 4.0915E−05 | / | / |
| T28 | 7.452E−11 | 1.3951E−08 | / |
| T56 | 5.3622E−09 | 1.2843E−07 | 0.017617329 |

Regarding the smooth appearance of the skin, a statistically significant increase was measured in 40% of volunteers after 7 days of use, in 50% of volunteers after 28 days of use, and in 65% of volunteers after 56 days of use. The decrease in skin roughness was measured in 45% of volunteers after 56 days of use (p<0.05).

The composition of the invention therefore allows roughness of the epidermis to be decreased and the smooth appearance thereof to be improved in significant manner on and after the first week.

Regarding wrinkle volume, the measured volumes are the following (see FIG. 16):

TABLE 14

| Day of measurement | Mean | Standard deviation |
|---|---|---|
| T0 | 4.33 | 0.94 |
| T7 | 4.22 | 0.85 |
| T28 | 4.13 | 0.84 |
| T56 | 4.09 | 0.84 |

Compared with the base value (TO), the volume of skin wrinkles was improved by:
2%, after 7 days of use of the product (p<0.05);
4%, after 28 days of use of the product (p<0.05); and
5%, after 56 days of use of the product (p<0.05).

There was also recorded a statistically significant reduction in the volume of skin wrinkles, of:
2% between the values at T7 (after 7 days' treatment) and T28 (after 28 days' treatment) (p<0.05);
3% between the values at T7 (after 7 days' treatment) and T56 (after 56 days' treatment) (p<0.05); and
1% between the values at T28 (after 28 days' treatment) and T56 (after 56 days' treatment) (p<0.05).

The statistical significance of the values is given below:

TABLE 15
p value (statistically significant for p < 0.05)

| | T0 | T7 | T28 |
|---|---|---|---|
| T7 | 0.01311924 | / | / |
| T28 | 0.00104163 | 0.03285423 | / |
| T56 | 0.00069357 | 0.01502898 | 0.026311258 |

The composition of the invention therefore allows the volume of wrinkles and fine lines to be significantly reduced on and after the first week.

An increase in skin radiance was measured to be statistically significant in 40% of volunteers after 7 days of use, in 50% of volunteers after 28 days of use and in 70% of volunteers after 56 days of use. A significant increase in even skin tone was measured in 35% of volunteers after 56 days of use.

The composition of the invention therefore allows the signs of ageing to be significantly reduced and in particular a statistically significant increase in moisturization of the skin, in skin elasticity, skin radiance, the smooth appearance of the skin, and a significant reduction in skin roughness and wrinkle volume.

### Example 3—Face Contour Serum

An example of cosmetic composition of the invention formulated as a serum for the face contour was tested in 20 women aged between 45 and 60 years. The formulation of the composition of the invention is given in Table 16:

TABLE 16
Face serum

| Ingredients (INCI | CAS | | Weight |
|---|---|---|---|
| name) | number | Role | percent |
| Aqua | 7732-18- | Solvent | q.s. to |
| | 5 | | 100% |
| Glycerin | 56-81-5 | Humectant, | 6.588000 |
| | | solvent | |
| Methylpropanediol | 2163-42- | solvent | 3.0 |
| | 0 | | |
| Isopropyl Myristate | 110-27-0 | Binder, emollient, | 2.0 |
| | | fragrance, solvent | |
| Nicotinamide | 1094-61- | Active cosmetic | 2.0 |
| Mononucleotide | 7 | substance | |
| Pentylene Glycol | 5343-92- | Solvent, emollient | 2.0 |
| | 0 | | |
| Squalane | 111-01-3 | Emollient | 1.5 |
| C14-C22 alcohols | / | Emulsion | 1.975 |
| | | stabilising | |
| Hydroxyethyl | 111286- | Emulsion | 0.925 |
| Acrylate/Sodium | 86-3 | stabilising, | |
| Acryloyldimethyl | | viscosity | |
| Taurate Copolymer | | controlling | |
| Maltodextrin | 9050-36- | Absorbent, binder, | 0.78 |
| | 6 | emulsion | |
| | | stabilising, film- | |
| | | forming, emollient | |
| PANTHENOL | 81-13-0 | Emollient | 0.5 |
| C12-20 Alkyl | / | Emulsifying, | 0.475 |
| Glucoside | | surfactant | |
| Leontopodium | / | Antioxidant, | 0.4 |
| Alpinum Callus | | humectant, skin | |
| Culture Extract | | protecting | |
| Squalane | 111-01-3 | Emollient | 0.357 |
| Glyceryl Caprylate | 26402- | Emollient, | 0.30 |
| | 26-6 | emulsifying | |
| Caprylic/Capric | 73398- | Emollient, solvent, | 0.16 |
| Triglyceride | 61-5 | fragrance | |
| Lecithin | 8002-43- | Emollient, | 0.145 |
| | 5 | emulsifying | |
| Sodium Hydroxide | 1310-73- | buffering | 0.111 |
| | 2 | | |
| Polysorbate 60 | 9005-67- | Chelating/buffering | 0.107 |
| | 8 | | |
| Sorbitan Isostearate | 71902- | Emulsifying | 0.051 |
| | 01-7 | | |
| Biosaccharide Gum- | 194237- | Emollient | 0.05 |
| 1 | 89-3 | | |
| Ethylhexylglycerin | 70445- | Emollient | 0.049950 |
| | 33-9 | | |
| Pistacia Lentiscus | 61789- | Film-forming | 0.0400 |
| Gum | 92-2 | | |
| Glucose | 50-99-7 | Humectant | 0.025000 |
| Citric Acid | 77-92-9 | Antioxidant | 0.0115 |
| Sodium Benzoate | 532-32-1 | Preserving | 0.006 |
| Xanthan Gum | 6132-04- | Binder, | 0.006 |
| | 3 | emulsifying, | |
| | | surfactant, | |
| | | viscosity | |
| | | controlling, gelling, | |
| | | emollient | |
| Magnolia Officinalis | / | Emollient | 0.002250 |
| Bark Extract | | | |
| Tocopherol | 10191- | Antioxidant, | 0.001550 |
| | 41-059- | emollient | |
| | 02-9 | | |
| Vitis Vinifera Seed | 84929- | Antioxidant, | 0.000450 |
| Extract | 27-1 | antimicrobial, UV | |
| | | absorbing, skin | |
| | | protecting | |

Different signs of ageing were assessed in the participants and in particular skin roughness via fringe projection, skin firmness and skin elasticity via Cutometry®, skin density via DermaScan® ultrasound, swelling of the lower eyelid, dark circles under the eye and tired appearance of the skin via clinical evaluation by an expert. These parameters were measured in patients at Day 0, at the time of inclusion in the study and before any treatment, at Day 7, at Day 28 in the middle of the study, and at Day 56 at the end of the study. The different parameters were also compared between the different measurement dates to assess the continuity of improvement. Wrinkle volume and assessment of skin roughness were evaluated with Antera 3D® software. The results were analysed with a Student's test on EXCEL software (Microsoft) and SAS 9.4.

Regarding moisturization, the measured values are the following (see FIG. 17):

TABLE 17

| Day of measurement | Mean | Standard deviation |
|---|---|---|
| T0 | 53.0 | 4.8 |
| T7 | 55.8 | 4.3 |
| T28 | 56.8 | 4.0 |
| T56 | 58.0 | 3.7 |

Compare with the base value (T0), I moisturization of the skin was increased by:
5%, after 7 days of use of the product 7 (p<0.05);
7%, after 28 days of use of the product (p<0.05); and
9%, after 56 days of use of the product (p<0.05).

There was also recorded a statistically significant increase in skin moisturization of:
2% between the values at T7 (after 7 days' treatment) and T28 (after 28 days' treatment) (p<0.05);
4% between the values at T7 (after 7 days' treatment) and T56 (after 56 days' treatment) (p<0.05); and
2% between the values at T28 (after 28 days' treatment) and T56 (after 56 days' treatment) (p<0.05).

The statistical significance of the values is given below:

TABLE 18
p value (statistically significant for p < 0.05)

| | T0 | T7 | T28 |
|---|---|---|---|
| T7 | 3.7498E−06 | / | / |
| T28 | 9.0486E−11 | 0.01815949 | / |
| T56 | 1.8226E−09 | 0.00022906 | 0.002889285 |

The composition of the invention therefore allows an improvement in moisturization of the epidermis on and after the first week.

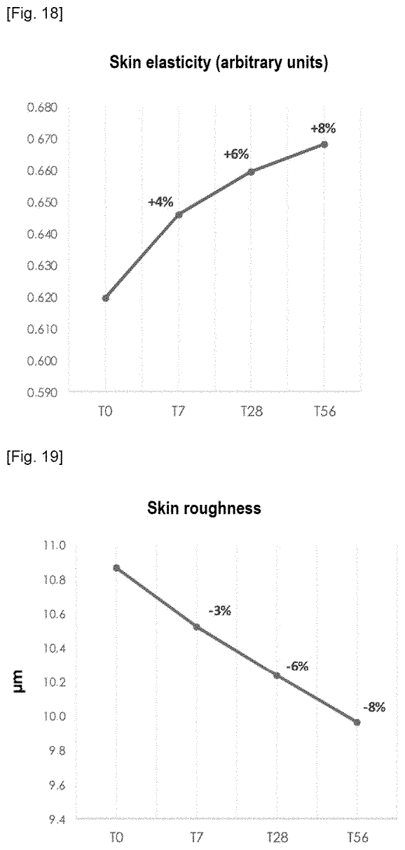

Regarding elasticity, the measured values are the following (see FIG. 18):

TABLE 19

| Day of measurement | Mean | Standard deviation |
|---|---|---|
| T0 | 0.620 | 0.034 |
| T7 | 0.646 | 0.037 |
| T28 | 0.660 | 0.031 |
| T56 | 0.668 | 0.031 |

Compared with the base value (TO), skin elasticity was increased by:
4%, after 7 days of use of the product (p<0.05);
6%, after 28 days of use of the product (p<0.05); and
8%, after 56 days of use of the product (p<0.05).

There was also recorded a statistically significant increase in skin elasticity of:
4% between the values at T7 (after 7 days' treatment) and T28 (after 28 days' treatment) (p<0.05);
3% between the values at T7 (after 7 days' treatment) and T56 (after 56 days' treatment) (p<0.05); and
1% between the values at T28 (after 28 days' treatment) and T56 (after 56 days' treatment) (p<0.05).

The statistical significance of the values is given below:

TABLE 20
p value (statistically significant for p < 0.05)

| | T0 | T7 | T28 |
|---|---|---|---|
| T7 | 7.23E−07 | / | / |
| T28 | 7.93E−10 | 0.000107 | / |
| T56 | 1.56E−10 | 1.88E−07 | 0.003712 |

The composition of the invention therefore allows elasticity of the epidermis to be significantly improved on and after the first week.

Regarding skin roughness, the measured values are the following (see FIG. 19):

TABLE 21

| Day of measurement | Mean | Standard deviation |
|---|---|---|
| T0 | 10.9 | 2.9 |
| T7 | 10.5 | 2.7 |
| T28 | 10.2 | 2.6 |
| T56 | 10.0 | 2.3 |

Compared with the base value (TO), skin roughness is improved by:
3%, after 7 days of use of the product (p<0.05);
6%, after 28 days of use of the product (p<0.05); and
8%, after 56 days of use of the product (p<0.05).

There was also recorded a statistically significant decrease in skin roughness of:
3% between the values at T7 (after 7 days' treatment) and T28 (after 28 days' treatment) (p<0.05);
5% between the values at T7 (after 7 day's treatment) and T56 (after 56 days' treatment) (p<0.05); and
7% between the values at T28 (after 28 days' treatment) and T56 (after 56 days' treatment) (p<0.05).

The statistical significance of the values is given below:

TABLE 22
p value (statistically significant for p < 0.05)

| | T0 | T7 | T28 |
|---|---|---|---|
| T7 | 0.00017 | / | / |
| T28 | 4.9E−07 | 4.26E−05 | / |
| T56 | 1.18E−05 | 0.000285 | 0.012686 |

Regarding skin roughness, a statistically significant decrease was measured in 25% of volunteers after 7 days of use, in 35% of volunteers after 28 days of use and in 50% of volunteers after 56 days of use.

Regarding the smooth appearance of the skin, a statistically significant increase was measured in 45% of volunteers after 7 days of use, in 50% of volunteers after 28 days of use and in 70% of volunteers after 56 days of use. The improvement in skin roughness was measured in 45% of volunteers after 56 days of use (p<0.05).

The composition of the invention therefore allows roughness of the epidermis to be reduced and the smooth appearance thereof to be significantly increased, on and after the first week.

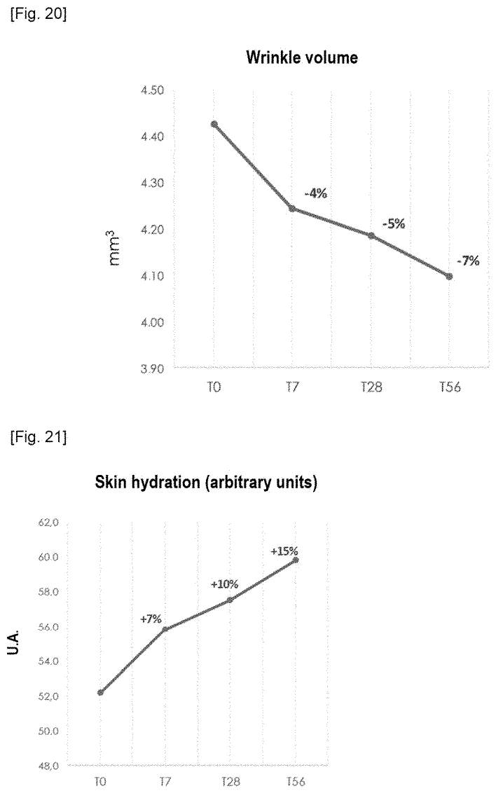

Regarding wrinkle volume, the measured values are the following (see FIG. 20):

TABLE 23

| Day of measurement | Mean | Standard deviation |
|---|---|---|
| T0 | 4.43 | 2.18 |
| T7 | 4.24 | 1.98 |
| T28 | 4.19 | 1.95 |
| T56 | 4.10 | 1.89 |

Compared with the base value (TO), wrinkle volume was reduced by:
4%, after 7 days of use of the product (p<0.05);
5%, after 28 days of use of the product; and
7%, after 56 days of use of the product (p<0.05).

There was also recorded a statistically significant reduction in the volume of skin wrinkles of:
1% between the values at T7 (after 7 days' treatment) and T28 (after 28 days' treatment) (p<0.05);
3% between the values at T7 (after 7 days' treatment) and T56 (after 56 days' treatment) (p<0.05); and
2% between the values at T28 (after 28 days' treatment) and T56 (after 56 days' treatment) (p<0.05).

The statistical significance of the values is given below:

TABLE 24
p value (statistically significant for p < 0.05)

| | T0 | T7 | T28 |
|---|---|---|---|
| T7 | 0.002633 | / | / |
| T28 | 0.000705 | 0.004432 | / |
| T56 | 0.000235 | 1.07E−05 | 0.000119 |

The composition of the invention therefore allows wrinkles and fine lines to be significantly reduced on and after the first week.

An increase in skin radiance was measured to be statistically significant in 35% of volunteers after 7 days of use, in 45% of volunteers after 28 days of use and in 60% of volunteers after 56 days of use. Significant even skin tone was measured in 35% of volunteers after 56 days of use.

The composition of the invention comprising NMN and an extract of Edelweiss therefore allows the signs of ageing to be significantly reduced, and in particular allows a statistically significant improvement in moisturization of the skin, in skin elasticity, skin radiance, the smooth appearance thereof, and significantly reduces skin roughness and wrinkle volume.

### Example 2—Eye Contour Serum

An example of a cosmetic composition of the invention formulated as an eye contour serum was tested in 20 women aged between 45 and 60 years. The formulation of the composition of the invention is given in the Table below:

TABLE 25
Eye contour serum

| Ingredients | CAS | | Weight |
|---|---|---|---|
| (INCI name) | number | Role | percent |
| Aqua | 7732-18-5 | Solvent | q.s. to |
| | | | 100% |
| Glycerin | 56-81-5 | Humectant, | 7.658800 |
| | | solvent | |
| Nicotinamide | 1094-61-7 | Active cosmetic | 2.0 |
| Mononucleotide | | substance | |
| Pentylene Glycol | 5343-92-0 | Solvent, emollient | 2.0 |
| Squalane | 111-01-3 | Emollient | 1.5 |
| C14-C22 alcohols | / | / | 1.185000 |
| Hydroxyethyl | 111286- | Emulsion | 0.8 |
| Acrylate/Sodium | 86-3 | stabilising, | |
| Acryloyldimethyl Taurate | | viscosity | |
| Copolymer | | controlling | |
| Polyacrylate | / | Emulsion | 0.8 |
| Crosspolymer-6 | | stabilising, | |
| | | viscosity | |
| | | controlling | |
| Glyceryl Stearate SE | 11099-07-3 | Emulsifying, | 0.5 |
| | | emollient | |
| Dicaprylyl Carbonate | 1680-31-5 | Emollient, | 0.499813 |
| Fagus Sylvatica Bud | 85251-65-6 | Astringent | 0.35 |
| Extract | | | |
| Glyceryl Caprylate | 26402-26-6 | Emollient, | 0.30 |
| | | emulsifying | |
| C12-20 Alkyl | / | Emulsifying, | 0.285 |
| Glucoside | | surfactant | |
| Allantoin | 97-59-6 | Lipid-replenishing, | 0.20 |
| | | skin protecting | |
| Diethylhexyl | 444811-29-4 | Skin protecting | 0.18 |
| Syringylidenemalonate | | | |
| Sodium Hydroxide | 1310-73-2 | Buffering | 0.13950 |
| Ethylhexylglycerin | 70445-33-9 | Emollient | 0.09990 |
| Benzyl Alcohol | 100-51-6 | Fragrance, | 0.060 |
| | | preserving, | |
| | | viscosity | |
| | | controlling, | |
| | | solvent | |
| Polysorbate 60 | 9005-67-8 | Chelating/ | 0.060 |
| | | buffering | |
| Sorbitan Isostearate | 71902-01-7 | Emulsifying | 0.060 |
| Leontopodium Alpinum | / | Antioxidant, | 0.04 |
| Callus Culture Extract | | humectant | |
| Chlorella Vulgaris | 223749- | Emollient | 0.0225 |
| Extract | 83-5 | | |
| Caprylic/Capric | 73398-61-5 | Emollient, solvent, | 2.0 |
| Triglyceride | | fragrance, lipid- | |
| | | replenishing | |
| Glucose | 50-99-7 | Humectant | 0.015 |
| Palmaria Palmata | 223751- | Skin protecting | 0.01 |
| Extract | 74-4 | | |
| Hydrolyzed Linseed | / | Emollient | 0.00950 |
| Extract | | | |
| Sodium Benzoate | 532-32-1 | Preserving agent | 0.0075 |
| Citric Acid | 77-92-9 | Antioxidant | 0.0061 |
| Benzoic Acid | 65-85-0 | Preserving agent | 0.004 |
| Xanthan Gum | 6132-04-3 | Binder, | 0.000600 |
| | | emulsifying, | |
| | | surfactant, | |
| | | viscosity | |
| | | controlling, | |
| | | gelling, lipid- | |
| | | replenishing | |
| Tocopherol | 10191-41-0 | Antioxidant, | 0.000288 |
| | 59-02-9 | Emollient | |

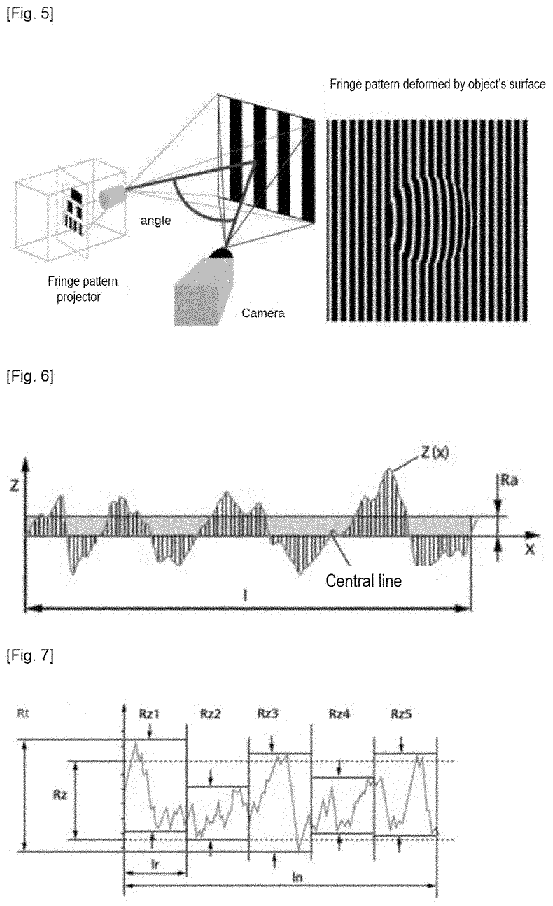

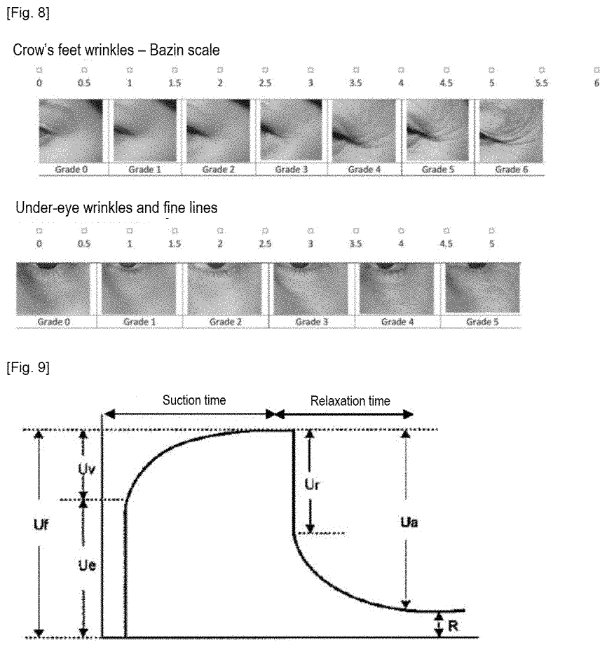

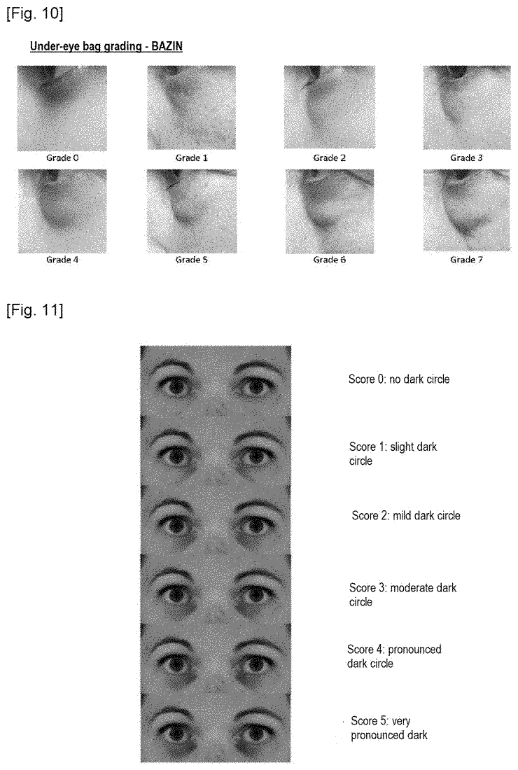

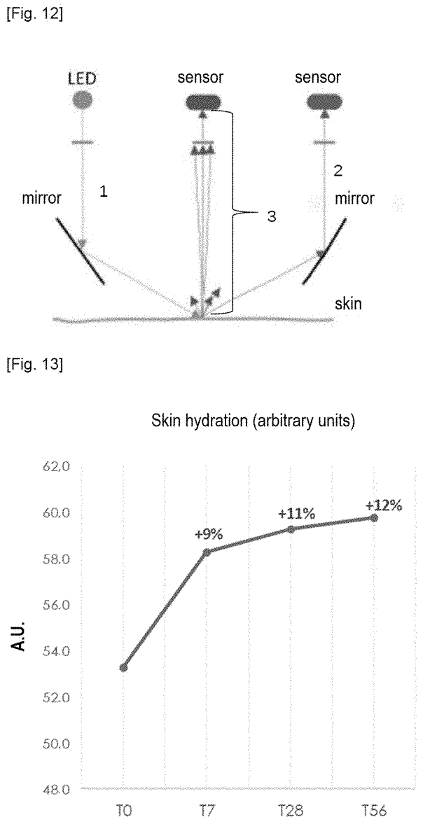

Different signs of ageing were assessed in the participants and in particular skin roughness via fringe projection (see FIG. 5), skin firmness and skin elasticity via Cutometry®, skin density via DermaScan® ultrasound, swelling of the lower eyelid also known as bags under the eyes, dark circles under the eye and tired appearance of the skin via clinical evaluation by an expert. These parameters were measured in the patients at Day 0 at the time of inclusion in the study and before any treatment, at Day 28 in the middle of the study, and at Day 56 at the end of the study. The different parameters were also compared between the different dates of measurement to assess continuity of improvement. Wrinkle volume and assessment of skin roughness were evaluated with Antera 3D® software. The results were analysed with a Student's test on EXCEL software (Microsoft) and SAS 9.4.

Regarding moisturization, the measured values are the following (see FIG. 21):

TABLE 26

| Day of measurement | Mean | Standard deviation |
|---|---|---|
| T0 | 52.2 | 3.0 |
| T7 | 55.8 | 2.0 |
| T28 | 57.5 | 2.1 |
| T56 | 59.8 | 2.6 |

Compared with the base value (TO), moisturizing of the skin increased by:
7%, after 7 days of use of the product (p<0.05);
10%, after 28 days of use of the product (p<0.05); and
15%, after 56 days of use of the product (p<0.05).

There was also recorded a statistically significant increase in skin moisturization of:
3% between the values at T7 (after 7 days' treatment) and T28 (after 28 days' treatment) (p<0.05); and
7% between the values at T7 (after 7 days' treatment) and T56 (after 56 days' treatment) (p<0.05); and
4% between the values at T28 (after 28 days' treatment) and T56 (after 56 days' treatment) (p<0.05).

The statistical significance of the values is given below:

TABLE 27
p value (statistically significant for p < 0.05)

| | T0 | T7 | T28 |
|---|---|---|---|
| T7 | 4.287E−08 | / | / |
| T28 | 1.621E−08 | 0.00027348 | / |
| T56 | 1.71E−10 | 3.3914E−08 | 2.63669E−05 |

The composition of the invention therefore allows moisturizing of the epidermis to be significantly improved on and after the first week.

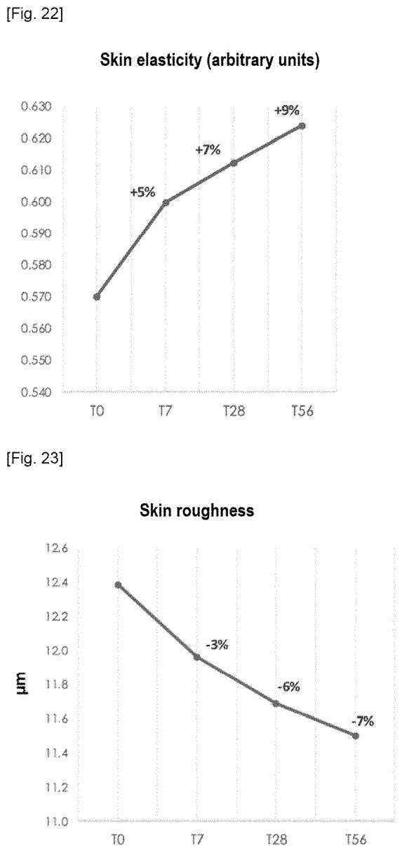

Regarding elasticity, the measured values are the following (see FIG. 22):

TABLE 28

| Day of measurement | Mean | Standard deviation |
|---|---|---|
| T0 | 0.570 | 0.058 |
| T7 | 0.600 | 0.060 |
| T28 | 0.612 | 0.051 |
| T56 | 0.624 | 0.049 |

Compared with the base value (TO), skin elasticity was increased by:
5%, after 7 days of use of the product (p<0.05);
7%, after 28 days of use of the product (p<0.05); and
9%, after 56 days of use of the product (p<0.05).

There was also recorded a statistically significant increase in skin elasticity of:
2% between the values at T7 (after 7 days' treatment) and T28 (after 28 days' treatment) (p<0.05); and
4% between the values at T7 (after 7 days' treatment) and T56 (after 56 days' treatment) (p<0.05); and
2% between the values at T28 (after 28 days' treatment) and T56 (after 56 days' treatment) (p<0.05).

The statistical significance of the values is given below:

TABLE 29
p value (statistically significant for p < 0.05)

| | T0 | T7 | T28 |
|---|---|---|---|
| T7 | 9.14E−07 | / | / |
| T28 | 1.84E−08 | 0.010169 | / |
| T56 | 1.29E−09 | 3.71E−05 | 0.00083 |

The composition of the invention therefore allows elasticity of the epidermis to be significantly improved on and after the first week.

Regarding skin roughness, the measured values are the following (see FIG. 23):

TABLE 30

| Day of measurement | Mean | Standard deviation |
|---|---|---|
| T0 | 12.4 | 2.0 |
| T7 | 12.0 | 1.9 |
| T28 | 11.7 | 1.8 |
| T56 | 11.5 | 1.7 |

Compare with the base value (T0), skin roughness was reduced by:
3%, after 7 days of use of the product (p<0.05);
6%, after 28 days of use of the product (p<0.05); and
7%, after 56 days of use of the product (p<0.05).

There was also recorded a statistically significant decrease in skin roughness, of:
2% between the values at T7 (after 7 days' treatment) and T28 (after 28 days' treatment) (p<0.05);
4% between the values at T7 (after 7 days' treatment) and T56 (after 56 days' treatment) (p<0.05); and
2% between the values at T28 (after 28 days' treatment) and T56 (after 56 days' treatment) (p<0.05).

The statistical significance of the values is given below:

TABLE 31
p value (statistically significant for p < 0.05)

| | T0 | T7 | T28 |
|---|---|---|---|
| T7 | 5.77E−05 | / | / |
| T28 | 3.41E−08 | 0.000731 | / |
| T56 | 8.47E−09 | 8.46E−07 | 2.14E−05 |

Regarding the smooth appearance of the skin, a statistically significant increase was measured in 45% of volunteers after 7 days of use, in 60% of volunteers after 28 days of use and in 75% of volunteers after 56 days of use. The reduction in skin roughness was measured in 50% of volunteers after 28 days of use and in 55% of volunteers after 56 days of use (p<0.05).

The composition of the invention therefore allows roughness of the epidermis at the eye contour to be reduced, and allows significant improvement in the smooth appearance thereof on and after the first week.

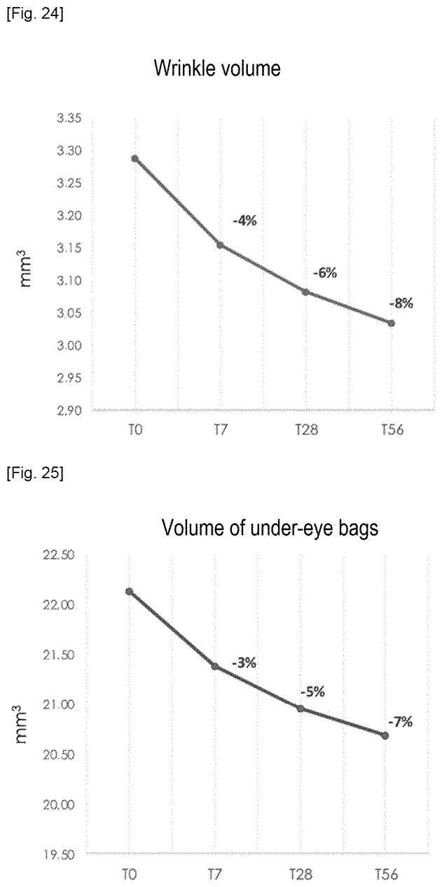

Regarding wrinkle volume, the measured values are the following (see FIG. 24):

TABLE 32

| Day of measurement | Mean | Standard deviation |
|---|---|---|
| T0 | 3.29 | 1.47 |
| T7 | 3.15 | 1.40 |
| T28 | 3.08 | 1.35 |
| T56 | 3.03 | 1.34 |

Compared with the base value (TO), the volume of skin wrinkles was improved by:
4%, after 7 days of use of the product (p<0.05);
4%, after 28 days of use of the product (p<0.05); and
5%, after 56 days of use of the product (p<0.05).

There was also recorded a statistically significant reduction in the volume of skin wrinkles, of:
2% between the values at T7 (after 7 days' treatment) and T28 (after 28 days' treatment) (p<0.05);
4% between the values at T7 (after 7 days' treatment) and T56 (after 56 days' treatment) (p<0.05); and
2% between the values at T28 (after 28 days' treatment) and T56 (after 56 days' treatment) (p<0.05).

The statistical significance of the values is given below:

TABLE 33
p value (statistically significant for p < 0.05)

| | T0 | T7 | T28 |
|---|---|---|---|
| T7 | 0.000603 | / | / |
| T28 | 7.16E−06 | 0.007166 | / |
| T56 | 1.27E−06 | 0.000159 | 0.000371 |

The composition of the invention therefore allows the volume of wrinkles and fine lines to be significantly reduced, on and after the first week.

An increase in skin radiance was measured to be statistically significant in 40% of volunteers after 7 days of use, in 60% of volunteers after 28 days of use and in 75% of volunteers after 56 days of use.

Even skin tone was measured to be significant in 35% of volunteers after 28 days of use and in 50% of volunteers after 56 days of use.

Regarding the volume of under-eye bags, the measured values are the following (see FIG. 25):

TABLE 34

| Day of measurement | Mean | Standard deviation |
|---|---|---|
| T0 | 22.1 | 4.6 |
| T7 | 21.4 | 4.1 |
| T28 | 21.0 | 4.0 |
| T56 | 20.7 | 3.9 |

Compared with the base value (TO), the volume of under-eye bags was improved by:
3%, after 7 days of use of the product (p<0.05);
5%, after 28 days of use of the product (p<0.05); and
7%, after 56 days of use of the product (p<0.05).

There was also recorded a statistically significant reduction in the volume of under-eye bags:
2% between the values at T7 (after 7 days' treatment) and T28 (after 28 days' treatment) (p<0.05);
3% between the values at T7 (after 7 days' treatment) and T56 (after 56 days' treatment) (p<0.05); and
1% between the values at T28 (after 28 days' treatment) and T56 (after 56 days' treatment) (p<0.05).

The statistical significance of the values is given below:

TABLE 35
p value (statistically significant for p < 0.05)

| | T0 | T7 | T28 |
|---|---|---|---|
| T7 | 0.0107571 | / | / |
| T28 | 0.000539 | 5.6059E−05 | / |
| T56 | 0.0001048 | 1.63605E−06 | 2.74E−06 |

A reduction in the visibility of under-eye bags was measured to be statistically significant in 40% of volunteers after 28 days of use, and in 55% of volunteers after 56 days of use.

The composition of the invention therefore allows the volume of bags under the eyes to be significantly reduced on and after the first week.

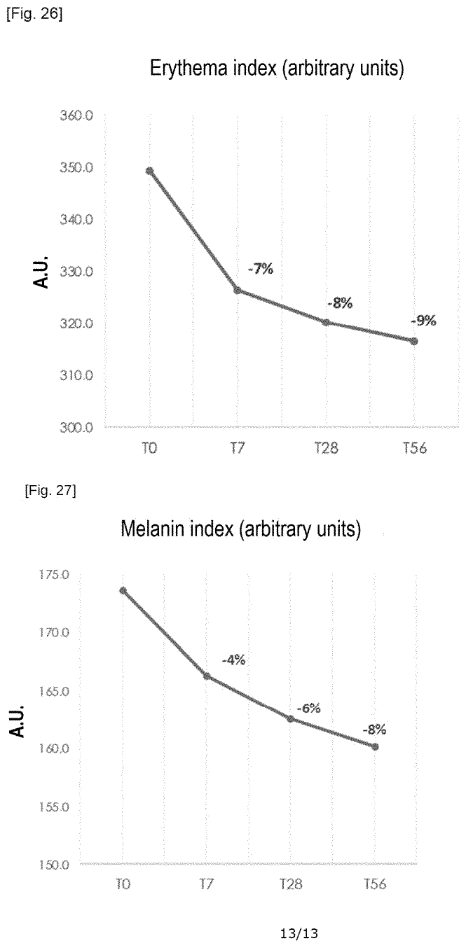

Regarding erythema under the eyes, assessed using a Mexameter® MX 18 following the manufacturer's instructions, the measured values are the following (see FIG. 26):

TABLE 36

| Day of measurement | Mean | Standard deviation |
|---|---|---|
| T0 | 349.3 | 58.5 |
| T7 | 326.3 | 49.6 |
| T28 | 320.2 | 50.0 |
| T56 | 316.5 | 49.6 |

Compared with the base value (TO), erythema of dark circles was improved by:
7%, after 7 days of use of the product (p<0.05);
8%, after 28 days of use of the product (p<0.05); and
9%, after 56 days of use of the product (p<0.05).

There was also recorded a statistically significant reduction in skin erythema, of:
2% between the values at T7 (after 7 days' treatment) and T28 (after 28 days' treatment) (p<0.05);
3% between the values at T7 (after 7 days' treatment) and T56 (after 56 days' treatment) (p<0.05); and
1% between the values at T28 (after 28 days' treatment) and T56 (after 56 days' treatment) (p<0.05).

The statistical significance of the values is given below:

TABLE 37
p value (statistically significant for p < 0.05)

| | T0 | T7 | T28 |
|---|---|---|---|
| T7 | 9E−05 | / | / |
| T28 | 2.87E−07 | 0.046357 | / |
| T56 | 1.94E−07 | 0.008644 | 0.004654 |

The composition of the invention therefore allows a significant reduction in erythema of under-eye bags, on and after the first week.

Regarding the melanin content of dark circles reflecting the colouring thereof, as assessed with the use of a Mexameter® MX 18 following the manufacturer's instructions, the measured values are the following (see FIG. 27):

TABLE 38

| Day of measurement | Mean | Standard deviation |
|---|---|---|
| T0 | 174 | 25 |
| T7 | 166 | 24 |
| T28 | 163 | 22 |
| T56 | 160 | 22 |

Compared with the base value (TO), colouring of dark circles was reduced by:
4%, after 7 days of use of the product (p<0.05);
6%, after 28 days of use of the product (p<0.05); and
8%, after 56 days of use of the product (p<0.05).

There was also recorded a statistically significant reduction in the volume of skin wrinkles, of:
2% between the values at T7 (after 7 days' treatment) and T28 (after 28 days' treatment) (p<0.05);
4% between the values at T7 (after 7 days' treatment) and T56 (after 56 days' treatment) (p<0.05); and
1% between the values at T28 (after 28 days' treatment) and T56 (after 56 days' treatment) (p<0.05).

The statistical significance of the values is given below:

TABLE 39
p value (statistically significant for p < 0.05)

| | T0 | T7 | T28 |
|---|---|---|---|
| T7 | 0.002475 | / | / |
| T28 | 1.1E−06 | 0.018411 | / |
| T56 | 4.19E−08 | 0.000848 | 0.001489 |

The composition of the invention therefore allows a significant reduction in erythema of under-eye bags, on and after the first week.

A reduction in the visibility of dark circles was measured to be statistically significant in 35% of volunteers after 7 days of use, in 45% of volunteers after 28 days of use, and in 55% of volunteers after 56 days of use. A significant increase in even skin tone was measured in 35% of volunteers after 56 days of use.

The composition of the invention therefore allows the signs of ageing to be significantly reduced, and in particular allows a statistically significant improvement in skin moisturization, skin elasticity, skin radiance, the smooth appearance thereof, and significantly reduces skin roughness, wrinkle volume, the volume of under-eye bags and the colouring of dark circles.

1-12. (canceled)

13. A cosmetic composition comprising nicotinamide mononucleotide (NMN), a derivative thereof, a precursor thereof or a salt thereof, or combinations thereof, to prevent or treat at least one sign of ageing.

14. The cosmetic composition as claimed in claim 13, wherein the precursor can be nicotinamide riboside (NR) and/or dihydronicotinamide riboside (NR—H).

15. The cosmetic composition as claimed in claim 13, wherein the derivative of NMN can be selected from among alpha nicotinamide mononucleotide (α-NMN), dihydronicotinamide mononucleotide (denoted NMN-H), the compound of formula (I): 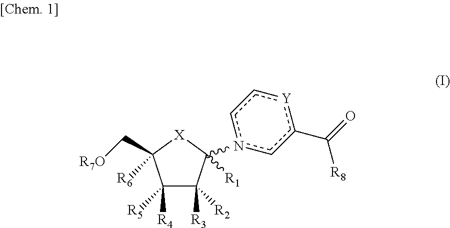
or one of the pharmaceutically acceptable stereoisomers, salts, hydrates, solvates or crystals thereof, in which:
X is selected from among O, CH₂, S, Se, CHF, CF₂ et C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ et R₅ are each independently selected from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from among a proteinogenic amino acid;
R₆ is selected from among H, azido, cyano, C₁-C₈ alkyl, C₁-C₈ thio-alkyl, C₁-C₈ heteroalkyl and OR; wherein R is selected from H and (C₁-C₈) alkyl;
R₇ is selected from among H, P(O)R₉R₁₀, P(S)R₉R₁₀ and 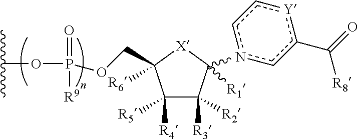
wherein n is an integer equal to 1 or 3; in which
R₉ and R₁₀ are each independently selected from among OH, OR₁₁, NHR₁₃, NR₁₃R₁₄, a (C₁-C₈) alkyl, a (C₂-C₈) alkenyl, a (C₂-C₈) alkynyl, a (C₃-C₁₀) cycloalkyl, a (C₅-C₁₂) aryl, (C₁-C₈) alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; and P(O)(OH)OP(O)(OH)₂, halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂ SO² wherein each of R₁₁ₐ is independently selected from among H and (C₁-C₆) alkyl and R_{11b} is independently selected from among OH, C₁-C₆ alkoxy, NH₂, NH(C₁-C₆ alkyl) or N(C₁-C₆ alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among a hydroxyl, (C₁-C₁₀) alkyl, (C₁-C₆) alkoxy, a halogen, a nitro, and a cyano; or
R₉ and R₁₀, together with the phosphorus atoms to which they are attached, form a 6-membered ring in which —R₉-R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
R₉ and R₁₀, together with the phosphorus atoms to which they are attached, form a 6-membered ring in which —R₉-R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃ and halogen; wherein R₁₃ and R₁₄ are each independently selected from among H, (C₁-C₈) alkyl and (C₁-C₈) alkyl aryl; and
R₁₃ and R₁₄ are each independently selected from among H, C₁-C₈ alkyl and C₁-C₈ alkyl aryl;
R₁₅ and R₁₆ are independently selected from among H, C₁-C₈alkyl and C₁-C₈alkyl aryl; and —CR_{B}R_{C}—C(O)—OR_{D} in which R_{B} and R_{C} are independently selected from among H, C₁-C₆ alkyl, C₁-C₆ alkoxy, benzyl, indolyl or imidazolyl, wherein the groups C₁-C₆ alkyl and C₁-C₆ alkoxy can optionally and each independently be substituted by one or more from among a halogen, an amino, amido, guanidyl, hydroxyl, thiol or carboxyl group, and the benzyl group is optionally substituted by one or more from among a halogen or hydroxyl group, or R_{B} and R_{C} together with the carbon atom to which they are attached form a C₃-C₆ cycloalkyl group optionally substituted by one or more from among a halogen, an amino, amido, guanidyl, hydroxyl, thiol or carboxyl group, and R_{D} is H, C₁-C₆ alkyl, C₂-C₆ alkenyl, C₂-C₆ alkynyl or C₃-C₆ cycloalkyl;
Y is selected from among CH, CH₂, C(CH₃)₂ and CCH₃; [symbol]
represents a single or double bond depending on Y; and [symbol]
represents an alpha or beta anomer depending on the position of R₁
or one of the stereoisomers, salts, hydrates, solvates or crystals thereof
or the compound of formula (Ia): 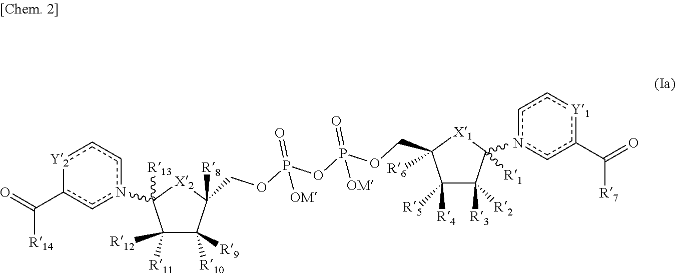
or one of the stereoisomers, salts, hydrates, solvates, or crystals thereof, in which
X′₁ and X′₂ are independently selected from among O, CH₂, S, Se, CHF, CF₂, et C═CH₂;
R′₁ and R′₁₃ are independently selected from among H, azido, cyano, a C₁-C₈ alkyl, a C₁-C₈ thio-alkyl, a C₁-C₈ heteroalkyl, and OR, wherein R is selected from H and a C₁-C₈ alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteogenic amino acid;
R′₆ and R′₈ are independently selected from among H, an azido, a cyano, a C₁-C₈ alkyl and OR, wherein R is selected from among H and a C₁-C₈ alkyl;
R′₇ and R′₁₄ are independently selected from among H, OR, NHR, NRR′, NH—NHR, SH, CN, N₃ and a halogen, wherein R and R′ are independently selected from among H and a (C₁-C₈) alkyl aryl;
Y′₁ and Y′₂ are independently selected from among CH, CH₂, C(CH₃)₂ or CCH³;
M′ is selected from among H or a suitable counter ion; [symbol]
represents a single or double bond depending on Y′₁ and Y′₂; and [symbol] represents an alpha or beta anomer depending on the position of R′₁ and R′₁₃;
and combinations thereof.

16. The cosmetic composition as claimed in claim 13, wherein the at least one sign of ageing is selected from among wrinkles, increased skin roughness, reduced thickness of the epidermis and dermis, reduced firmness of the epidermis and dermis, reduced elasticity of the epidermis and dermis, diminished skin radiance, swelling of the lower eyelid, erythema of under-eye skin, dark circles and combinations thereof.

17. The cosmetic composition as claimed in claim 13, comprising between 0.01 weight % and 30 weight %, preferably between 0.1 weight % and 10 weight %, more preferably between 1 weight % and 5 weight %, and most preferably about 2 weight % of NMN, a derivative thereof, a precursor thereof or a salt thereof, relative to the total weight of the composition.

18. The cosmetic composition as claimed in claim 13, further comprising an extract of Edelweiss.

19. The cosmetic composition as claimed in claim 18, comprising between 0.001 weight % and 10 weight %, preferably between 0.005 weight % and 5 weight %, more preferably between 0.01 weight % and 1 weight %, and further preferably about 0.04 weight % of extract of Edelweiss, relative to the total weight of the composition.

20. The cosmetic composition as claimed in claim 13, further comprising hyaluronic acid, preferably sodium hyaluronate.

21. The cosmetic composition as claimed in claim 20, comprising between 0.01 weight % and 5 weight % of hyaluronic acid, preferably between 0.05 weight % and 2 weight % of hyaluronic acid, more preferably between 0.1 weight % and 1 weight % of hyaluronic acid, most preferably about 0.35% of hyaluronic acid by weight of the composition.

22. The cosmetic composition as claimed in claim 13, in the form of a gel, solution, water-in-oil emulsion, oil-in-water emulsion, oil, cream, ointment, liposomal dispersion or a liniment.

23. The cosmetic composition as claimed in claim 13, having a pH of between 4 and 9, preferably between 5 and 7.

24. The cosmetic composition as claimed in claim 13, selected from among a serum, universal cream, day cream, night cream, eye contour cream, makeup removing lotion, makeup removing milk, makeup removing oil, makeup removing balm, cosmetic mask, toning lotion, exfoliating product, cleansing gel, cleansing foam, cleansing milk, cleansing lotion, preferably a serum.

25. A non-therapeutic cosmetic method comprising the application to the skin surface of the cosmetic composition as claimed in claim 13.
